(19) United States
(12) Patent Application Publication
(10) Pub. No.: US 2026/0315447 A1
(43) Pub. Date: Oct. 15, 2026

(54) COMBINATION THERAPY WITH A MTOR INHIBITOR AND A MULTI-TYROSINE KINASE INHIBITOR FOR TREATING SOFT TISSUE SARCOMA

(71) Applicant: ADDi Bioscience, Inc., Morristown, NJ (US)

(72) Inventors: Lee D. CRANMER, Seattle, WA (US); Neil P. DESAI, Pacific Palisades, CA (US); Anita N. SCHMID, Berkeley Heights, NJ (US)

(21) Appl. No.: 19/485,634

(22) PCT Filed: May 23, 2024
(86) PCT No.: PCT/US2024/030782

§ 371 Date: Nov. 18, 2025

Related U.S. Application Data
(60) Provisional application No. 63/468,758, filed on May 24, 2023.

(51) Int. Cl. A61K 31/439; A61K 9/51; A61K 31/506; A61P 35/00

(52) U.S. Cl. CPC A61K 31/439; A61K 9/5169; A61K 31/506; A61P 35/00

(57) ABSTRACT

The present invention relates to methods and kits for the treatment of breast cancer based on hormone receptor status of progesterone receptor and estrogen receptor comprising the administration of a taxane alone, in combination with at least one other and other therapeutic agents, as well as other treatment modalities useful in the treatment of breast cancer. In particular, the invention relates to the use of nanoparticles comprising paclitaxel and albumin (such as Abraxane®) either alone or in combination with other chemotherapeutic agents or radiation, which may be used for the treatment of breast cancer which does not express estrogen receptor and/or progesterone receptor.

### CROSS-REFERENCE TO RELATED APPLICATIONS

This application claims the priority benefit of U.S. Provisional Patent Application Ser. No. 63/468,758, filed on May 24, 2023, the entire contents of which are incorporated herein by reference for all purposes.

### FIELD OF THE INVENTION

The present application, in certain aspects, pertains to methods and compositions for the treatment of a soft-tissue sarcoma using a composition comprising nanoparticles comprising an mTOR inhibitor and an albumin in combination with a multi-tyrosine kinase inhibitor.

### BACKGROUND OF THE INVENTION

Soft-tissue sarcomas may present with many different histological subtypes and are generally difficult to treat. Soft-tissue sarcomas have a low 5-year survival rate, especially following development of metastases, and many chemotherapeutic agents have not provided meaningful patient benefit. For example, single agent pazopanib was not found to provide a significant difference in overall survival, as compared to a placebo control, to patients having a soft-tissue sarcoma.

mTOR inhibitors have found wide applications in treating diverse pathological conditions such as solid tumors, hematological malignancies, organ transplantation, restenosis, and rheumatoid arthritis. One such example is sirolimus (INN/USAN), also known as rapamycin—an immunosuppressant drug used to prevent rejection in organ transplantation. Sirolimus-eluting stents were approved in the United States to treat coronary restenosis. Additionally, sirolimus has been demonstrated as an effective inhibitor of tumor growth in various cell lines and animal models. Other limus drugs, such as analogs of sirolimus, have been designed to improve the pharmacokinetic and pharmacodynamic properties of sirolimus. For example, Temsirolimus was approved in the United States and Europe for the treatment of renal cell carcinoma. Everolimus was approved in the United States for treatment of advanced breast cancer, pancreatic neuroendocrine tumors, advanced renal cell carcinoma, and subependymal giant cell astrocytoma (SEGA) associated with Tuberous Sclerosis. The mode of action of sirolimus is to bind the cytosolic protein FK-binding protein 12 (FKBP12), and the sirolimus-FKBP12 complex in turn inhibits the mTOR pathway by directly binding to the mTOR Complex 1 (mTORC1).

Albumin-based nanoparticle compositions have been developed as a drug delivery system for delivering substantially water insoluble drugs. See, for example, U. S. Pat. Nos. 5,916,596; 6,506,405; 6,749,868, and 6,537,579, 7,820,788, and 7,923,536. Abraxane®, an albumin stabilized nanoparticle formulation of paclitaxel, was approved in the United States in 2005 and subsequently in various other countries for treating metastatic breast cancer. It was recently approved for treating non-small cell lung cancer in the United States, and has also shown therapeutic efficacy in various clinical trials for treating difficult-to-treat cancers such as bladder cancer and melanoma. Albumin derived from human blood has been used for the manufacture of Abraxane® as well as various other albumin-based nanoparticle compositions. Albumin-based nanoparticle composition comprising sirolimus, e.g., nab-sirolimus or Fyarrao®, are known, e.g., U.S. Pat. Nos. 8,911,786 and 11,497,737.

Despite the efficacious use of single agent pazopanib or nab-sirolimus in certain cancer contexts, there remains a continuing need in the art for advanced treatments of certain cancers including the difficult-to-treat soft-tissue sarcomas.

### BRIEF DESCRIPTION OF THE DRAWINGS

FIG. 1 shows a swimmer plot of total treatment duration for evaluable patients administered nab-sirolimus in combination with pazopanib.

FIG. 2 shows a waterfall plot of responses of 18 evaluable patients administered nab-sirolimus in combination with pazopanib.

### BRIEF SUMMARY OF THE INVENTION

The present application, in certain aspects, provides a method of treating a soft-tissue sarcoma (STS) in an individual in need thereof, the method comprising administering to the individual: (a) a composition comprising nanoparticles comprising an mTOR inhibitor and an albumin; and (b) a multi-tyrosine kinase inhibitor, wherein the multi-tyrosine kinase inhibitor (e.g., pazopanib) is administered in an amount of about 1 mg to about 2500 mg, such as about 200 mg to about 800 mg.

In some embodiments according to any of the methods described herein, the soft-tissue sarcoma is selected from the group consisting of spindle cell sarcoma, solitary fibrous tumor, leiomyosarcoma, myxofibrosarcoma, undifferentiated pleomorphic sarcoma, melanocytic schwannoma, and pleomorphic rhabdomyosarcoma.

In other aspects, provided is a method of treating a soft-tissue sarcoma in an individual in need thereof, wherein the soft-tissue sarcoma is selected from the group consisting of spindle cell sarcoma, solitary fibrous tumor, leiomyosarcoma, myxofibrosarcoma, undifferentiated pleomorphic sarcoma, melanocytic schwannoma, and pleomorphic rhabdomyosarcoma, the method comprising administering to the individual: (a) a composition comprising nanoparticles comprising an mTOR inhibitor and an albumin; and (b) a multi-tyrosine kinase inhibitor (e.g., pazopanib).

In some embodiments according to any of the methods described herein, the leiomyosarcoma is uterine leiomyosarcoma. In some embodiments, the leiomyosarcoma is non-uterine leiomyosarcoma.

In some embodiments according to any of the methods described herein, the soft-tissue sarcoma is locally advanced, advanced, malignant, advanced malignant, or metastatic. In some embodiments according to any of the methods described herein, the soft-tissue sarcoma is relapsed, refractory, or resistant to a prior treatment. In some embodiments according to any of the methods described herein, the prior treatment comprises a composition comprising nanoparticles comprising an mTOR inhibitor and an albumin.

In some embodiments according to any of the methods described herein, the amount of the mTOR inhibitor in the mTOR inhibitor nanoparticle composition administered to the individual is from about 10 mg/m² to about 150 mg/m². In some embodiments according to any of the methods described herein, the amount of the mTOR inhibitor in the mTOR inhibitor nanoparticle composition administered to the individual is about 10 mg/m² to about 60 mg/m². In some embodiments according to any of the methods described herein, the amount of the mTOR inhibitor in the mTOR inhibitor nanoparticle composition administered to the individual is about 30 mg/m², 45 mg/m², or 60 mg/m².

In some embodiments according to any of the methods described herein, the mTOR inhibitor nanoparticle composition is administered once or twice out of every 3 weeks. In some embodiments according to any of the methods described herein, the mTOR inhibitor nanoparticle composition is administered on day 1 of a 21-day cycle. In some embodiments according to any of the methods described herein, the mTOR inhibitor nanoparticle composition is administered on days 1 and 8 of a 21-day cycle.

In some embodiments according to any of the methods described herein, the mTOR inhibitor is a limus drug. In some embodiments, the limus drug is sirolimus.

In some embodiments according to any of the methods described herein, the average diameter of the nanoparticles in the composition is no greater than about 150 nm. In some embodiments according to any of the methods described herein, the average diameter of the nanoparticles in the composition is no greater than about 120 nm. In some embodiments according to any of the methods described herein, the weight ratio of the albumin to the mTOR inhibitor in the nanoparticle composition is no greater than about 9:1. In some embodiments according to any of the methods described herein, the nanoparticles comprise the mTOR inhibitor associated with the albumin. In some embodiments according to any of the methods described herein, the nanoparticles comprise the mTOR inhibitor coated with the albumin.

In some embodiments according to any of the methods described herein, the mTOR inhibitor nanoparticle composition is administered intravenously.

In some embodiments according to any of the methods described herein, the multi-tyrosine kinase inhibitor inhibits two or more of the following: vascular endothelial growth factor receptor (VEGFR)-1, -2, or -3, platelet endothelial growth factor receptor (PDGFR)-α or -β, interleukin-2 receptor-inducible T-cell kinase (ITK), leukocyte-specific protein tyrosine kinase (LCK), colony-stimulating factor-1 receptor (c-fms), fibroblast growth factor receptors (FGFR)-1, -3, or -4, or stem cell factor receptor c-Kit.

In some embodiments according to any of the methods described herein, the multi-tyrosine kinase inhibitor is pazopanib or a pharmaceutically acceptable salt thereof. In some embodiments according to any of the methods described herein, the multi-tyrosine kinase inhibitor is pazopanib hydrochloride. In some embodiments according to any of the methods described herein, the multi-tyrosine kinase inhibitor is administered in an amount of about 400 mg. In some embodiments according to any of the methods described herein, the multi-tyrosine kinase inhibitor is administered daily or every other day. In some embodiments according to any of the methods described herein, the multi-tyrosine kinase inhibitor is administered orally. In some embodiments according to any of the methods described herein, the multi-tyrosine kinase inhibitor is administered without food.

In some embodiments according to any of the methods described herein, when the mTOR inhibitor nanoparticle composition is administered, the multi-tyrosine kinase inhibitor is administered concurrently. In some embodiments according to any of the methods described herein, when the mTOR inhibitor nanoparticle composition is administered, the multi-tyrosine kinase inhibitor is administered sequentially. In some embodiments according to any of the methods described herein, when the mTOR inhibitor nanoparticle composition is administered, the multi-tyrosine kinase inhibitor is administered simultaneously.

In some embodiments according to any of the methods described herein, the mTOR inhibitor in the mTOR inhibitor nanoparticle composition is administered intravenously in an amount of about 30 mg/m² on day 1 of a 21-day cycle, and wherein the multi-tyrosine kinase inhibitor is administered orally in an amount of about 400 mg daily.

In some embodiments according to any of the methods described herein, the individual is human.

The disclosures of all publications, patents, patent applications and published patent applications referred to herein are hereby incorporated herein by reference in their entirety.

### DETAILED DESCRIPTION OF THE INVENTION

The present application provides, in certain aspects, treatments for soft-tissue sarcomas (e.g., spindle cell sarcoma, solitary fibrous tumor, or leiomyosarcoma) comprising a combination of (a) a composition comprising nanoparticles comprising an mTOR inhibitor (such as a limus drug, e.g., sirolimus or a derivative thereof) and an albumin (e.g., nab-sirolimus); and (b) a multi-tyrosine kinase inhibitor (e.g., pazopanib).

The subject matter the present application is based, at least in part, on the finding that nab-sirolimus, a composition comprising nanoparticles comprising the mTOR inhibitor sirolimus, and an albumin, and (b) the multi-tyrosine kinase inhibitor pazopanib, are safe and effective in treating patients having soft-tissue sarcomas. Soft-tissue sarcomas, including spindle cell sarcoma, solitary fibrous tumor, or leiomyosarcoma, are very difficult to treat cancers and patients diagnosed with such cancers face poor prognoses even with available approved treatments. For example, soft-tissue sarcoma patients administered single agent pazopanib treatment exhibit about a 6% response rate, and did not have a meaningful improvement in overall survival rate as compared to a placebo control. As discussed in more detail in the Examples section, dosages and schedules of the combination of nab-sirolimus and pazopanib were found to be safe and effective in providing benefit to patients having a soft-tissue sarcoma. For instance, 16 out of 18 evaluable patients treated with the combination of nab-sirolimus and pazopanib exhibited stable disease or partial response. A finding of stable disease is significant in the context of soft-tissue sarcoma, which is an especially difficult-to-treat cancer. Moreover, the combination of nab-sirolimus and pazopanib provide a long-term patient benefit with some patients experiencing progression free survival for over a year. Such findings represent a significant advancement in the treatment of soft-tissue sarcomas as even a result of stable disease for a relative short period of time, not to mention the extended period reported in the Examples section, is a substantial improvement over existing soft-tissue sarcoma treatments.

Thus, provided herein, in certain aspects, is a method of treating a soft-tissue sarcoma in an individual in need thereof, the method comprising administering to the individual: (a) a composition comprising nanoparticles comprising an mTOR inhibitor and an albumin; and (b) a multi-tyrosine kinase inhibitor (e.g., pazopanib). In some embodiments, the soft-tissue sarcoma is selected from the group consisting of spindle cell sarcoma, solitary fibrous tumor, leiomyosarcoma, myxofibrosarcoma, undifferentiated pleomorphic sarcoma, melanocytic schwannoma, and pleomorphic rhabdomyosarcoma. In some embodiments, the multi-kinase inhibitor (e.g., pazopanib) is administered at about 1 mg to about 2500 mg, such as about 200 mg to about 800 mg, such as about 400 mg. In some embodiments, the composition comprising nanoparticles comprising the mTOR inhibitor and the albumin is administered at about 10 mg/m² to about 100 mg/m², such as about 30 mg/m², about 45 mg/m², or about 60 mg/m². In some embodiments, the composition comprising nanoparticles comprising the mTOR inhibitor and the albumin is administered once every three weeks, such as on day 1 of a 21-day cycle. In some embodiments, the composition comprising nanoparticles comprising the mTOR inhibitor and the albumin is administered twice every three weeks, such as on days 1 and 8 of a 21-day cycle. In some embodiments, the multi-kinase inhibitor (e.g., pazopanib) is orally administered at about 400 mg every day, and the composition comprising nanoparticles comprising the mTOR inhibitor and the albumin is intravenously administered at about 30 mg/m² every three weeks, such as on day 1 of a 21-day cycle.

In other aspects, provided herein is a method of treating a soft-tissue sarcoma in an individual in need thereof, the method comprising administering to the individual: (a) a composition comprising nanoparticles comprising an mTOR inhibitor and an albumin; and (b) a multi-tyrosine kinase inhibitor (e.g., pazopanib), wherein the multi-tyrosine kinase inhibitor is administered at about 1 mg to about 2500 mg, such as about 200 mg to about 800 mg, such as about 400 mg. In some embodiments, the soft-tissue sarcoma is selected from the group consisting of spindle cell sarcoma, solitary fibrous tumor, leiomyosarcoma, myxofibrosarcoma, undifferentiated pleomorphic sarcoma, melanocytic schwannoma, and pleomorphic rhabdomyosarcoma. In some embodiments, the composition comprising nanoparticles comprising the mTOR inhibitor and the albumin is administered at about 10 mg/m² to about 100 mg/m², such as about 30 mg/m², 45 mg/m², or 60 mg/m². In some embodiments, the composition comprising nanoparticles comprising the mTOR inhibitor and the albumin is administered once every three weeks, such as on day 1 of a 21-day cycle. In some embodiments, the composition comprising nanoparticles comprising the mTOR inhibitor and the albumin is administered twice every three weeks, such as on days 1 and 8 of a 21-day cycle. In some embodiments, the multi-kinase inhibitor (e.g., pazopanib) is orally administered at about 400 mg every day, and the composition comprising nanoparticles comprising the mTOR inhibitor and the albumin is intravenously administered at about 30 mg/m² every three weeks, such as on day 1 of a 21-day cycle.

In other aspects, provided is a method of treating a soft-tissue sarcoma in an individual in need thereof, wherein the soft-tissue sarcoma is selected from the group consisting of spindle cell sarcoma, solitary fibrous tumor, leiomyosarcoma, myxofibrosarcoma, undifferentiated pleomorphic sarcoma, melanocytic schwannoma, and pleomorphic rhabdomyosarcoma, the method comprising administering to the individual: (a) a composition comprising nanoparticles comprising an mTOR inhibitor and an albumin; and (b) a multi-tyrosine kinase inhibitor (e.g., pazopanib). In some embodiments, the multi-kinase inhibitor is administered at about 1 mg to about 2500 mg, such as about 200 mg to about 800 mg, such as 400 mg. In some embodiments, the composition comprising nanoparticles comprising the mTOR inhibitor and the albumin is administered at about 10 mg/m² to about 100 mg/m², such as about 30 mg/m², about 45 mg/m², or about 60 mg/m². In some embodiments, the composition comprising nanoparticles comprising the mTOR inhibitor and the albumin is administered once every three weeks, such as on day 1 of a 21-day cycle. In some embodiments, the composition comprising nanoparticles comprising the mTOR inhibitor and the albumin is administered twice every three weeks, such as on days 1 and 8 of a 21-day cycle. In some embodiments, the multi-kinase inhibitor (e.g., pazopanib) is orally administered at about 400 mg every day, and the composition comprising nanoparticles comprising the mTOR inhibitor and the albumin is intravenously administered at about 30 mg/m² every three weeks, such as on day 1 of a 21-day cycle.

### I. Definitions

As used herein “nab” stands for nanoparticle albumin-bound, and “nab-sirolimus” is an albumin stabilized nanoparticle formulation of sirolimus. nab-sirolimus is also known as nab-rapamycin, which has been previously described. See, for example, U.S. Pat. Nos. 8,911,786 and 11,497,737, each of which is incorporated herein by reference in their entirety.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, reducing recurrence rate of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. In some embodiments, the treatment reduces the severity of one or more symptoms associated with cancer by at least about any of 10%, 20%, 30%, 40%, 50%, 60%, 70%, 80%, 90%, 95% or 100% compared to the corresponding symptom in the same subject prior to treatment or compared to the corresponding symptom in other subjects not receiving the treatment. Also encompassed by “treatment” is a reduction of pathological consequence of cancer. The methods of the invention contemplate any one or more of these aspects of treatment.

The terms “recurrence,” “relapse” or “relapsed” refers to the return of a cancer or disease after clinical assessment of the disappearance of disease. A diagnosis of distant metastasis or local recurrence can be considered a relapse.

The term “refractory” or “resistant” refers to a cancer or disease that has not responded to treatment.

As used herein, “delaying” the development of cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of subjects. Cancer development can be detectable using standard methods, including, but not limited to, computerized axial tomography (CAT scan), Magnetic Resonance Imaging (MRI), ultrasound, clotting tests, arteriography, biopsy, urine cytology, and cystoscopy. Development may also refer to cancer progression that may be initially undetectable and includes occurrence, recurrence, and onset.

The term “effective amount” used herein refers to an amount of a compound or composition sufficient to treat a specified disorder, condition or disease such as ameliorate, palliate, lessen, and/or delay one or more of its symptoms. In reference to cancer, an effective amount comprises an amount sufficient to cause a tumor to shrink and/or to decrease the growth rate of the tumor (such as to suppress tumor growth) or to prevent or delay other unwanted cell proliferation in cancer. In some embodiments, an effective amount is an amount sufficient to delay development of cancer. In some embodiments, an effective amount is an amount sufficient to prevent or delay recurrence. In some embodiments, an effective amount is an amount sufficient to reduce recurrence rate in the individual. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; (vii) reduce recurrence rate of tumor, and/or (viii) relieve to some extent one or more of the symptoms associated with the cancer.

As is understood in the art, an “effective amount” or “amount” may be in one or more doses, i.e., a single dose or multiple doses may be required to achieve the desired treatment endpoint. An effective amount may be considered in the context of administering one or more therapeutic agents, and a nanoparticle composition (e.g., a composition including sirolimus and an albumin) may be considered to be given in an effective amount if, in conjunction with one or more other agents, a desirable or beneficial result may be or is achieved. The components (e.g., the first and second therapies) in a combination therapy of the invention may be administered sequentially, simultaneously, or concurrently using the same or different routes of administration for each component. Thus, an effective amount of a combination therapy includes an amount of the first therapy and an amount of the second therapy that when administered sequentially, simultaneously, or concurrently produces a desired outcome.

“In conjunction with” or “in combination with” refers to administration of one treatment modality in addition to another treatment modality, such as administration of a nanoparticle composition described herein in addition to administration of the other agent to the same individual under the same treatment plan. As such, “in conjunction with” or “in combination with” refers to administration of one treatment modality before, during or after delivery of the other treatment modality to the individual.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy is contained in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U. S. Food and Drug administration.

As used herein, the term “individual” refers to a mammal and includes, but is not limited to, human, bovine, horse, feline, canine, rodent, rat, mouse, dog, or primate. In some embodiments, the individual is a human individual.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Those skilled in the art will recognize that several embodiments are possible within the scope and spirit of the present disclosure. The following description illustrates the disclosure and, of course, should not be construed in any way as limiting the scope of the inventions described herein.

### II. Methods of Treatment

Provided herein are methods for the treatment of soft-tissue sarcoma in an individual in need thereof, the methods comprising administering to the individual (a) a composition comprising nanoparticles comprising an mTOR inhibitor (such as a limus drug, e.g., sirolimus or a derivative thereof) and an albumin; and (b) a multi-tyrosine kinase inhibitor (e.g., pazopanib).

In some embodiments, provided is a method of treating a soft-tissue sarcoma in an individual in need thereof, the method comprising administering to the individual: (a) a composition comprising nanoparticles comprising an mTOR inhibitor and an albumin (e.g., nab-sirolimus); and (b) pazopanib. In some embodiments, the soft-tissue sarcoma is selected from the group consisting of spindle cell sarcoma, solitary fibrous tumor, leiomyosarcoma, myxofibrosarcoma, undifferentiated pleomorphic sarcoma, melanocytic schwannoma, and pleomorphic rhabdomyosarcoma. In some embodiments, pazopanib is administered at about 1 mg to about 2500 mg, such as about 200 mg to about 800 mg, such as about 400 mg. In some embodiments, pazopanib is administered orally. In some embodiments, pazopanib is administered daily. In some embodiments, the composition comprising nanoparticles comprising the mTOR inhibitor and the albumin is administered at about 10 mg/m² to about 100 mg/m², such as about 30 mg/m². In some embodiments, the composition comprising nanoparticles comprising the mTOR inhibitor and the albumin is administered intravenously. In some embodiments, the composition comprising nanoparticles comprising the mTOR inhibitor and the albumin is administered every three weeks. In some embodiments, pazopanib is administered at about 400 mg orally every day, and the composition comprising nanoparticles comprising the mTOR inhibitor and the albumin is administered at about 30 mg/m² intravenously every three weeks, such as on day 1 of a 21-day cycle.

In some embodiments, the soft-tissue sarcoma is selected from the group consisting of spindle cell sarcoma, solitary fibrous tumor, leiomyosarcoma, myxofibrosarcoma, undifferentiated pleomorphic sarcoma, melanocytic schwannoma, and pleomorphic rhabdomyosarcoma. In some embodiments, the leiomyosarcoma is uterine leiomyosarcoma. In some embodiments, the leiomyosarcoma is non-uterine leiomyosarcoma.

In some embodiments, the soft-tissue sarcoma is locally advanced, advanced, malignant, advanced malignant, or metastatic. In some embodiments, the soft-tissue sarcoma is relapsed, refractory, or resistant to a prior treatment. In some embodiments, the prior treatment comprises a composition comprising nanoparticles comprising an mTOR inhibitor and an albumin.

The methods provided herein are applicable to all stages of soft-tissue sarcoma, including stages, I, II, III, and IV, according to the American Joint Committee on Cancer (AJCC) staging groups. In some embodiments, the soft-tissue sarcoma is an early stage cancer, non-metastatic cancer, primary cancer, advanced cancer, locally advanced cancer, metastatic cancer, cancer in remission, cancer in an adjuvant setting, or cancer in a neoadjuvant setting. In some embodiments, the soft-tissue sarcoma is localized resectable, localized unresectable, or unresectable. In some embodiments, the soft-tissue sarcoma is localized resectable or borderline resectable.

In some embodiments of any of the methods described herein, the composition comprising nanoparticles comprising an mTOR inhibitor and an albumin and the multi-tyrosine kinase inhibitor are administered in effective amounts to effect a treatment of a soft-tissue sarcoma.

### A. Dosing and Methods of Administration

The dose of the mTOR inhibitor nanoparticle composition (such as sirolimus/albumin nanoparticle composition) administered to an individual (e.g., a human) may vary with the particular composition, the method of administration, and the particular stage of tumor being treated. The amount should be sufficient to produce a desirable response, such as a therapeutic or prophylactic response against the tumor. In some embodiments, the amount of mTOR inhibitor (such as a limus drug, e.g., sirolimus or a derivative thereof) in the composition is below the level that induces a toxicological effect (e.g., an effect above a clinically acceptable level of toxicity) or is at a level where a potential side effect can be controlled or tolerated when the mTOR inhibitor nanoparticle composition is administered to the individual.

In some embodiments, when the mTOR inhibitor nanoparticle composition (such as sirolimus/albumin nanoparticle composition) is administered, the mTOR inhibitor nanoparticle composition is administered to the individual simultaneously with a multi-tyrosine kinase inhibitor (e.g., pazopanib). For example, the mTOR inhibitor nanoparticle compositions and a multi-tyrosine kinase inhibitor (e.g., pazopanib) are administered with a time separation of no more than about 15 minutes, such as no more than about any of 10, 5, or 1 minutes. In one example, wherein the compounds are in solution, simultaneous administration can be achieved by administering a solution containing the combination of compounds. In another example, simultaneous administration of separate solutions or compositions, one of which contains the mTOR inhibitor nanoparticle composition (such as sirolimus/albumin nanoparticle composition) and the other of which contains a multi-tyrosine kinase inhibitor (e.g., pazopanib), can be employed. In one example, simultaneous administration can be achieved by administering a composition containing the combination of compounds. In another example, simultaneous administration can be achieved by administering two separate compositions, one comprising the mTOR inhibitor nanoparticle composition (such as sirolimus/albumin nanoparticle composition administered intravenously) and the other comprising a multi-tyrosine kinase inhibitor (e.g., pazopanib administered orally). In some embodiments, simultaneous administration of the mTOR inhibitor (such as a limus drug, e.g., sirolimus or a derivative thereof) in the nanoparticle composition and a multi-tyrosine kinase inhibitor (e.g., pazopanib) can be combined with supplemental doses of the mTOR inhibitor and/or the multi-tyrosine kinase inhibitor (e.g., pazopanib).

In other embodiments, the mTOR inhibitor nanoparticle composition (such as sirolimus/albumin nanoparticle composition) and a multi-tyrosine kinase inhibitor (e.g., pazopanib) are not administered simultaneously. In some embodiments, the mTOR inhibitor nanoparticle composition (such as sirolimus/albumin nanoparticle composition) is administered before a multi-tyrosine kinase inhibitor (e.g., pazopanib). In other embodiments, the multi-tyrosine kinase inhibitor (e.g., pazopanib) is administered before the mTOR inhibitor nanoparticle composition (such as sirolimus/albumin nanoparticle composition). The time difference in non-simultaneous administrations can be greater than 1 minute, five minutes, 10 minutes, 15 minutes, 30 minutes, 45 minutes, 60 minutes, two hours, three hours, six hours, nine hours, 12 hours, 24 hours, 36 hours, or 48 hours. In other embodiments, the first administered compound is provided time to take effect on the patient before the second administered compound is administered. In some embodiments, the difference in time does not extend beyond the time for the first administered compound to complete its effect in the patient, or beyond the time the first administered compound is completely or substantially eliminated or deactivated in the patient.

In some embodiments, the administration of the mTOR inhibitor nanoparticle composition (such as sirolimus/albumin nanoparticle composition) and a multi-tyrosine kinase inhibitor (e.g., pazopanib) are concurrent, i.e., the administration period of the mTOR inhibitor nanoparticle composition and that of the multi-tyrosine kinase inhibitor (e.g., pazopanib) overlap with each other. In some embodiments, the mTOR inhibitor nanoparticle composition (such as sirolimus/albumin nanoparticle composition) is administered for at least one cycle (for example, at least any of 2, 3, 4, 5, 6, 7, 8, 9, 10, 11, 12, 13, 14 15, 16, 17, 18, 19, or 20 cycles) prior to the administration of a multi-tyrosine kinase inhibitor (e.g., pazopanib). In some embodiments, the multi-tyrosine kinase inhibitor (e.g., pazopanib) is administered for at least any of one, two, three, or four weeks. In some embodiments, the administration of the mTOR inhibitor nanoparticle composition (such as sirolimus/albumin nanoparticle composition) and a multi-tyrosine kinase inhibitor (e.g., pazopanib) are initiated at about the same time (for example, within any one of 1 day, 2 days, 3 days, 4 days, 5 days, 6 days, 1 week, 2 weeks, or 3 weeks). In some embodiments, the administration of the mTOR inhibitor nanoparticle composition (such as sirolimus/albumin nanoparticle composition) and a multi-tyrosine kinase inhibitor (e.g., pazopanib) are terminated at about the same time (for example, within any one of 1 day, 2 days, 3 days, 4 days, 5 days, 6 days, 1 week, 2 weeks, or 3 weeks). In some embodiments, the administration of a multi-tyrosine kinase inhibitor (e.g., pazopanib) continues (for example for about any one of 1, 2, 3, 4, 5, 6, 7, 8, 9, 10, 11, or 12 months) after the termination of the administration of the mTOR inhibitor nanoparticle composition (such as sirolimus/albumin nanoparticle composition). In some embodiments, the administration of a multi-tyrosine kinase inhibitor (e.g., pazopanib) is initiated after (for example after about any one of 1, 2, 3, 4, 5, 6, 7, 8, 9, 10, 11, or 12 months) the initiation of the administration of the mTOR inhibitor nanoparticle composition (such as sirolimus/albumin nanoparticle composition). In some embodiments, the administrations of the mTOR inhibitor nanoparticle composition (such as sirolimus/albumin nanoparticle composition) and a multi-tyrosine kinase inhibitor (e.g., pazopanib) are initiated and terminated at about the same time. In some embodiments, the administrations of the mTOR inhibitor nanoparticle composition (such as sirolimus/albumin nanoparticle composition) and a multi-tyrosine kinase inhibitor (e.g., pazopanib) are initiated at about the same time and the administration of the multi-tyrosine kinase inhibitor (e.g., pazopanib) continues (for example for about any one of 1, 2, 3, 4, 5, 6, 7, 8, 9, 10, 11, or 12 months) after the termination of the administration of the mTOR inhibitor nanoparticle composition. In some embodiments, the administration of the mTOR inhibitor nanoparticle composition (such as sirolimus/albumin nanoparticle composition) and a multi-tyrosine kinase inhibitor (e.g., pazopanib) stop at about the same time and the administration of the multi-tyrosine kinase inhibitor (e.g., pazopanib) is initiated after (for example after about any one of 1, 2, 3, 4, 5, 6, 7, 8, 9, 10, 11, or 12 months) the initiation of the administration of the mTOR inhibitor nanoparticle composition.

In some embodiments, the multi-tyrosine kinase inhibitor (e.g., pazopanib) is administered daily. In some embodiments, the administration of a multi-tyrosine kinase inhibitor (e.g., pazopanib) is reduced for a patient consideration, such as an adverse event. In some embodiments, the reduction in administration of a multi-tyrosine kinase inhibitor (e.g., pazopanib) is to every other day. In some embodiments, the reduction in administration of a multi-tyrosine kinase inhibitor (e.g., pazopanib) is for only a portion of the treatment, e.g., until the individual recovers (at least partially) from the adverse event.

In some embodiments, the administration of the composition comprising an mTOR inhibitor and an albumin and a multi-tyrosine kinase inhibitor (e.g., pazopanib) continues for at least any of 1, 2, 3, 4, 5, 6, 7, 8, 9, 10, 11, 12, 13, 14 15, 16, 17, 18, 19, or 20 cycles.

In some embodiments, the administration of the mTOR inhibitor nanoparticle composition (such as sirolimus/albumin nanoparticle composition) and a multi-tyrosine kinase inhibitor (e.g., pazopanib) are non-concurrent. For example, in some embodiments, the administration of the mTOR inhibitor nanoparticle composition (such as sirolimus/albumin nanoparticle composition) is terminated before a multi-tyrosine kinase inhibitor (e.g., pazopanib) is administered. In some embodiments, the administration of a multi-tyrosine kinase inhibitor (e.g., pazopanib) is terminated before the mTOR inhibitor nanoparticle composition (such as sirolimus/albumin nanoparticle composition) is administered. The time period between these two non-concurrent administrations can range from about two to eight weeks, such as about four weeks.

The dosing frequency of the mTOR inhibitor nanoparticle composition (such as sirolimus/albumin nanoparticle composition) and a multi-tyrosine kinase inhibitor (e.g., pazopanib) may be adjusted over the course of the treatment, based on the judgment of the administering physician. When administered separately, the mTOR inhibitor nanoparticle composition (such as sirolimus/albumin nanoparticle composition) and a multi-tyrosine kinase inhibitor (e.g., pazopanib) can be administered at different dosing frequency or intervals. For example, the mTOR inhibitor nanoparticle composition (such as sirolimus/albumin nanoparticle composition) can be administered once every three weeks, while the multi-tyrosine kinase inhibitor (e.g., pazopanib) can be administered more or less frequently, e.g., daily. In some embodiments, a sustained continuous release formulation of the nanoparticle and/or a multi-tyrosine kinase inhibitor (e.g., pazopanib) may be used. Various formulations and devices for achieving sustained release are known in the art. A combination of the administration configurations described herein can also be used.

The mTOR inhibitor nanoparticle composition (such as sirolimus/albumin nanoparticle composition) and a multi-tyrosine kinase inhibitor (e.g., pazopanib) can be administered using the same route of administration or different routes of administration. In some embodiments (for both simultaneous and sequential administrations), the mTOR inhibitor (such as a limus drug, e.g., sirolimus or a derivative thereof) in the mTOR inhibitor nanoparticle composition and a multi-tyrosine kinase inhibitor (e.g., pazopanib) are administered at a predetermined ratio.

The doses required for the mTOR inhibitor (such as a limus drug, e.g., sirolimus or a derivative thereof) in the mTOR inhibitor nanoparticle composition and/or a multi-tyrosine kinase inhibitor (e.g., pazopanib) may (but not necessarily) be the same or lower than what is normally required when each agent is administered alone. Thus, in some embodiments, a subtherapeutic amount of the mTOR inhibitor (such as a limus drug, e.g., sirolimus or a derivative thereof) in the mTOR inhibitor nanoparticle composition and/or a multi-tyrosine kinase inhibitor (e.g., pazopanib). “Subtherapeutic amount” or “subtherapeutic level” refer to an amount that is less than the therapeutic amount, that is, less than the amount normally used when the mTOR inhibitor nanoparticle composition (such as sirolimus/albumin nanoparticle composition) and/or a multi-tyrosine kinase inhibitor (e.g., pazopanib) are administered alone. The reduction may be reflected in terms of the amount administered at a given administration and/or the amount administered over a given period of time (reduced frequency). In some embodiments, the method comprises administering a composition comprising nanoparticles comprising an mTOR inhibitor and an albumin at a dosage of less than about 100 mg/m², such as about any of 90 mg/m², 80 mg/m², 70 mg/m², 60 mg/m², 50 mg/m², 40 mg/m², 30 mg/m², 20 mg/m², or 10 mg/m². In some embodiments, the method comprises administering a multi-tyrosine kinase inhibitor (e.g., pazopanib) at a dosage of less than about 800 mg, such as about any of 700 mg, 600 mg, 500 mg, 400 mg, 300 mg, 200 mg, or 100 mg.

In some embodiments, enough of a multi-tyrosine kinase inhibitor (e.g., pazopanib) is administered so as to allow reduction of the normal dose of the mTOR inhibitor (such as a limus drug, e.g., sirolimus or a derivative thereof) in the mTOR inhibitor nanoparticle composition required to affect the same degree of treatment by at least about any of 5%, 10%, 20%, 30%, 50%, 60%, 70%, 80%, 90%, or more. In some embodiments, enough of the mTOR inhibitor (such as a limus drug, e.g., sirolimus or a derivative thereof) in the mTOR inhibitor nanoparticle composition is administered so as to allow reduction of the normal dose of the a multi-tyrosine kinase inhibitor (e.g., pazopanib) required to affect the same degree of treatment by at least about any of 5%, 10%, 20%, 30%, 50%, 60%, 70%, 80%, 90%, or more.

In some embodiments, the dose of both the mTOR inhibitor (such as a limus drug, e.g., sirolimus or a derivative thereof) in the mTOR inhibitor nanoparticle composition and a multi-tyrosine kinase inhibitor (e.g., pazopanib) are reduced as compared to the corresponding normal dose of each when administered alone. In some embodiments, both the mTOR inhibitor (such as a limus drug, e.g., sirolimus or a derivative thereof) in the mTOR inhibitor nanoparticle composition and a multi-tyrosine kinase inhibitor (e.g., pazopanib) are administered at a subtherapeutic, i.e., reduced, level. In some embodiments, the dose of the mTOR inhibitor (such as a limus drug, e.g., sirolimus or a derivative thereof) in the mTOR inhibitor nanoparticle composition and/or a multi-tyrosine kinase inhibitor (e.g., pazopanib) is substantially less than the established maximum toxic dose (MTD). For example, the dose of the mTOR inhibitor nanoparticle composition (such as sirolimus/albumin nanoparticle composition) and/or a multi-tyrosine kinase inhibitor (e.g., pazopanib) is less than about 50%, 40%, 30%, 20%, or 10% of the MTD.

A combination of the administration configurations described herein can be used. The combination therapy methods described herein may be performed alone or in conjunction with another therapy, such as surgery, radiation, gene therapy, immunotherapy, bone marrow transplantation, stem cell transplantation, hormone therapy, targeted therapy, cryotherapy, ultrasound therapy, photodynamic therapy, and/or chemotherapy and the like. Additionally, a person having a greater risk of developing the soft-tissue sarcoma may receive treatments to inhibit and/or delay the development of the disease.

As will be understood by those of ordinary skill in the art, in some embodiments, the appropriate doses of second agents will be approximately those already employed in clinical therapies wherein a multi-tyrosine kinase inhibitor (e.g., pazopanib) is administered alone or in combination with other chemotherapeutic agents. Variation in dosage will likely occur depending on the condition being treated. As described above, in some embodiments, the second chemotherapeutic agent may be administered at a reduced level.

In some embodiments, the amounts of the mTOR inhibitor nanoparticle composition (such as sirolimus/albumin nanoparticle composition) and a multi-tyrosine kinase inhibitor (e.g., pazopanib) are below the levels that induce a toxicological effect (i.e., an effect above a clinically acceptable level of toxicity) or are at a level where a potential side effect can be controlled or tolerated when the mTOR inhibitor nanoparticle composition and a multi-tyrosine kinase inhibitor (e.g., pazopanib) are administered to the individual.

In some embodiments, the amount of the mTOR inhibitor nanoparticle composition (such as sirolimus/albumin nanoparticle composition) is close to a maximum tolerated dose (MTD) of the composition following the same dosing regimen when administered with a multi-tyrosine kinase inhibitor (e.g., pazopanib). In some embodiments, the amount of the mTOR inhibitor nanoparticle composition (such as sirolimus/albumin nanoparticle composition) is more than about any of 80%, 90%, 95%, or 98% of the MTD when administered with a multi-tyrosine kinase inhibitor (e.g., pazopanib).

As described herein, in some embodiments, reference to amounts of a composition comprising nanoparticles comprising an mTOR inhibitor and an albumin are based on the amount of the mTOR inhibitor therein. In some embodiments, the amount of an mTOR inhibitor (such as a limus drug, e.g., sirolimus) in the mTOR inhibitor nanoparticle composition is about any of 25 mg/m², 30 mg/m², 45 mg/m², 50 mg/m², 56 mg/m², 60 mg/m², 75 mg/m², 80 mg/m², 90 mg/m², 100 mg/m², 120 mg/m², 160 mg/m², 175 mg/m², 180 mg/m², 200 mg/m², 210 mg/m², 220 mg/m², 250 mg/m², 260 mg/m², 300 mg/m², 350 mg/m², 400 mg/m², 500 mg/m², 540 mg/m², 750 mg/m², 1000 mg/m², or 1080 mg/m² mTOR inhibitor. In some embodiments, the mTOR inhibitor nanoparticle composition includes less than about any of 350 mg/m², 300 mg/m², 250 mg/m², 200 mg/m², 150 mg/m², 120 mg/m², 100 mg/m², 90 mg/m², 50 mg/m², or 30 mg/m² mTOR inhibitor (such as a limus drug, e.g., sirolimus). In some embodiments, the amount of the mTOR inhibitor (such as a limus drug, e.g., sirolimus) per administration is less than about any of 40 mg/m², 39 mg/m², 38 mg/m², 37 mg/m², 36 mg/m², 35 mg/m², 34 mg/m², 33 mg/m², 32 mg/m², 31 mg/m², 30 mg/m², 29 mg/m², 28 mg/m², 27 mg/m², 26 mg/m², 25 mg/m², 24 mg/m², 23 mg/m², 22 mg/m², 21 mg/m², 20 mg/m², 19 mg/m², 18 mg/m², 17 mg/m², 16 mg/m², 15 mg/m², 14 mg/m², 13 mg/m², 12 mg/m², 11 mg/m², 10 mg/m², 9 mg/m², 8 mg/m², 7 mg/m², 6 mg/m², 5 mg/m², 4 mg/m², 3 mg/m², 2 mg/m², or 1 mg/m². In some embodiments, the mTOR inhibitor (such as a limus drug, e.g., sirolimus) in the mTOR inhibitor nanoparticle composition is included in any of the following ranges: about 1 to about 5 mg/m², about 5 to about 10 mg/m², about 10 to about 25 mg/m², about 25 to about 50 mg/m², about 50 to about 75 mg/m², about 75 to about 100 mg/m², about 100 to about 125 mg/m², about 125 to about150 mg/m², about150 to about 175 mg/m², about175 to about 200 mg/m², about 200 to about 225 mg/m², about 225 to about 250 mg/m², about 250 to about 300 mg/m², about 300 to about 350 mg/m², or about 350 to about 400 mg/m². In some embodiments, the mTOR inhibitor (such as a limus drug, e.g., sirolimus) in the mTOR inhibitor nanoparticle composition is about 30 to about 300 mg/m², such as about 100 to about 150 mg/m², about 120 mg/m², about 130 mg/m², or about 140 mg/m². In some embodiments, the amount of the mTOR inhibitor nanoparticle composition is administered every four weeks (e.g., day 1 of a 28-day cycle). In some embodiments, the amount of the mTOR inhibitor nanoparticle composition is administered every three weeks (e.g., day 1 of a 21-day cycle). In some embodiments, the amount of the mTOR inhibitor nanoparticle composition is administered every two weeks (e.g., day 1 of a 14-day cycle). In some embodiments, the amount of the mTOR inhibitor nanoparticle composition is administered weekly. In some embodiments, the amount of the mTOR inhibitor nanoparticle composition is administered weekly every 2 out of 3 weeks. In some embodiments, the amount of the mTOR inhibitor nanoparticle composition is on days 8 and 15 of a 21-day cycle, days 1 or 8 of a 21-day cycle, days 15 and 21 or a 21-day cycle, days 1 and 15 of a 21-day cycle, or days 1 and 21 of a 21-day cycle.

In some embodiments, the multi-tyrosine kinase inhibitor (e.g., pazopanib) is administered at about 1 mg to about 2,500 mg, including any of about 100 mg to about 900 mg, about 200 mg to about 800 mg, about 200 mg to about 700 mg, about 200 mg to about 600 mg, or about 200 mg to about 400 mg. In some embodiments, the multi-tyrosine kinase inhibitor (e.g., pazopanib) is administered at about 2,500 mg or less, such as about any of 2,250 mg or less, 2,000 mg or less, 1,750 mg or less, 1,500 mg or less, 1,250 mg or less, 1,000 mg or less, 750 mg or less, 700 mg or less, 650 mg or less, 600 mg or less, 550 mg or less, 500 mg or less, 450 mg or less, 400 mg or less, 350 mg or less, 300 mg or less, 250 mg or less, 200 mg or less, 150 mg or less, or 100 mg or less. In some embodiments, the multi-tyrosine kinase inhibitor (e.g., pazopanib) is administered at about any of 2,500 mg, 2,400 mg, 2,300 mg, 2,200 mg, 2,100 mg, 2,000 mg, 1,900 mg, 1,800 mg, 1,700 mg, 1,600 mg, 1,500 mg, 1,400 mg, 1,300 mg, 1,200 mg, 1,100 mg, 1,000 mg, 900 mg, 800 mg, 750 mg, 700 mg, 650 mg, 600 mg, 550 mg, 500 mg, 450 mg, 400 mg, 350 mg, 300 mg, 250 mg, 200 mg, 150 mg, or 100 mg. In some embodiments, the multi-tyrosine kinase inhibitor (e.g., pazopanib) is administered at about 400 mg.

In some embodiments, the dosing frequencies for the administration of the mTOR inhibitor nanoparticle composition (such as sirolimus/albumin nanoparticle composition) include, but are not limited to, daily, every two days, every three days, every four days, every five days, every six days, weekly without break, three out of four weeks (such as on days 1, 8, and 15 of a 28-day cycle), once every three weeks, once every two weeks, or two out of three weeks. In some embodiments, the mTOR inhibitor nanoparticle composition (such as sirolimus/albumin nanoparticle composition) is administered about once every 2 weeks, once every 3 weeks, once every 4 weeks, once every 6 weeks, or once every 8 weeks. In some embodiments, the mTOR inhibitor nanoparticle composition (such as sirolimus/albumin nanoparticle composition) is administered at least about any of 1×, 2×, 3×, 4×, 5×, 6×, or 7× (i.e., daily) a week. In some embodiments, the intervals between each administration are less than about any of 6 months, 3 months, 1 month, 20 days, 15, days, 14 days, 13 days, 12 days, 11 days, 10 days, 9 days, 8 days, 7 days, 6 days, 5 days, 4 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week.

In some embodiments, the dosing frequency is once every two days for one time, two times, three times, four times, five times, six times, seven times, eight times, nine times, ten times, or eleven times. In some embodiments, the dosing frequency is once every two days for five times. In some embodiments, the mTOR inhibitor (such as a limus drug, e.g., sirolimus or a derivative thereof) is administered over a period of at least ten days, wherein the interval between each administration is no more than about two days, and wherein the dose of the mTOR inhibitor at each administration is about 0.25 mg/m² to about 250 mg/m², about 0.25 mg/m² to about 150 mg/m², about 0.25 mg/m² to about 75 mg/m², such as about 0.25 mg/m² to about 25 mg/m², or about 25 mg/m² to about 50 mg/m².

The administration of the mTOR inhibitor nanoparticle composition (such as sirolimus/albumin nanoparticle composition) can be extended over an extended period of time, such as from about a month up to about seven years. In some embodiments, the mTOR inhibitor nanoparticle composition (such as sirolimus/albumin nanoparticle composition) is administered over a period of at least about any of 2, 3, 4, 5, 6, 7, 8, 9, 10, 11, 12, 18, 24, 30, 36, 48, 60, 72, or 84 months.

In some embodiments, the dosage of an mTOR inhibitor (such as a limus drug, e.g., sirolimus or a derivative thereof) in a nanoparticle composition can be in the range of 5-400 mg/m² when given on a 3-week schedule, or 5-250 mg/m² (such as 80-150 mg/m², for example 100-120 mg/m²) when given on a weekly schedule. For example, the amount of an mTOR inhibitor (such as a limus drug, e.g., sirolimus or a derivative thereof) is about 60 to about 300 mg/m² (e.g., about 260 mg/m²) on a 3-week schedule.

In some embodiments, the exemplary dosing schedules for the administration of the mTOR inhibitor nanoparticle composition (such as sirolimus/albumin nanoparticle composition) include, but are not limited to, 100 mg/m², weekly, without break; 10 mg/m² weekly, 3 out of four weeks (such as on days 1, 8, and 15 of a 28-day cycle); 45 mg/m² weekly, 3 out of four weeks (such as on days 1, 8, and 15 of a 28-day cycle); 75 mg/m² weekly, 3 out of four weeks (such as on days 1, 8, and 15 of a 28-day cycle); 100 mg/m², weekly, 3 out of 4 weeks; 125 mg/m², weekly, 3 out of 4 weeks; 125 mg/m², weekly, 2 out of 3 weeks; 130 mg/m², weekly, without break; 175 mg/m², once every 2 weeks; 260 mg/m², once every 2 weeks; 260 mg/m², once every 3 weeks; 180-300 mg/m², every three weeks; 60-175 mg/m², weekly, without break; 20-150 mg/m² twice a week; and 150-250 mg/m² twice a week. The dosing frequency of the mTOR inhibitor nanoparticle composition (such as sirolimus/albumin nanoparticle composition) may be adjusted over the course of the treatment based on the judgment of the administering physician.

In some embodiments, the individual is treated for at least about any of one, two, three, four, five, six, seven, eight, nine, or ten treatment cycles.

The mTOR inhibitor nanoparticle composition (such as sirolimus/albumin nanoparticle composition) described herein allow infusion of the mTOR inhibitor nanoparticle composition to an individual over an infusion time that is shorter than about 24 hours. For example, in some embodiments, the mTOR inhibitor nanoparticle composition (such as sirolimus/albumin nanoparticle composition) is administered over an infusion period of less than about any of 24 hours, 12 hours, 8 hours, 5 hours, 3 hours, 2 hours, 1 hour, 30 minutes, 20 minutes, or 10 minutes. In some embodiments, the mTOR inhibitor nanoparticle composition (such as sirolimus/albumin nanoparticle composition) is administered over an infusion period of about 30 minutes.

In some embodiments, the exemplary dose of the mTOR inhibitor (in some embodiments a limus drug, e.g., sirolimus) in the mTOR inhibitor nanoparticle composition includes, but is not limited to, about any of 10 mg/m², 20 mg/m², 30 mg/m², 40 mg/m², 50 mg/m², 60 mg/m², 75 mg/m², 80 mg/m², 90 mg/m², 100 mg/m², 120 mg/m², 160 mg/m², 175 mg/m², 200 mg/m², 210 mg/m², 220 mg/m², 260 mg/m², and 300 mg/m². For example, the dosage of an mTOR inhibitor (such as a limus drug, e.g., sirolimus or a derivative thereof) in a nanoparticle composition can be in the range of about 20-400 mg/m² when given on a 3-week schedule, or about 10-250 mg/m² when given on a weekly schedule.

In some embodiments, the dosage of an mTOR inhibitor (such as a limus drug, e.g., sirolimus) is about 100 mg to about 400 mg, for example about 100 mg, about 200 mg, about 300 mg, or about 400 mg. In some embodiments, the limus drug is administered at about 100 mg weekly, about 200 mg weekly, about 300 mg weekly, about 100 mg twice weekly, or about 200 mg twice weekly. In some embodiments, the administration is further followed by a monthly maintenance dose (which can be the same or different from the weekly doses).

In some embodiments when the mTOR nanoparticle composition is administered intravenously, the dosage of an mTOR inhibitor (such as a limus drug, e.g., sirolimus) in a nanoparticle composition can be in the range of about 30 mg to about 400 mg. The mTOR inhibitor nanoparticle composition (such as sirolimus/albumin nanoparticle composition) described herein allow infusion of the mTOR inhibitor nanoparticle composition to an individual over an infusion time that is shorter than about 24 hours. For example, in some embodiments, the mTOR inhibitor nanoparticle composition (such as sirolimus/albumin nanoparticle composition) is administered over an infusion period of less than about any of 24 hours, 12 hours, 8 hours, 5 hours, 3 hours, 2 hours, 1 hour, 30 minutes, 20 minutes, or 10 minutes. In some embodiments, the mTOR inhibitor nanoparticle composition (such as sirolimus/albumin nanoparticle composition) is administered over an infusion period of about 30 minutes to about 40 minutes.

In some embodiments, the exemplary dosing schedules for the administration of a multi-tyrosine kinase inhibitor include daily or every other day. In some embodiments, the exemplary dosing schedule for the administration of a multi-tyrosine kinase inhibitor is daily. In some embodiments, the exemplary dosing schedule for the administration of a multi-tyrosine kinase inhibitor is 1, 2, 3, 4, 5, 6 or 7 times a week. In some embodiments, the multi-tyrosine kinase inhibitor (e.g., pazopanib) is suitable for oral administration.

An mTOR inhibitor nanoparticle composition (such as sirolimus/albumin nanoparticle composition) and a multi-tyrosine kinase inhibitor (e.g., pazopanib), in pure form or in an appropriate pharmaceutical composition, can be administered via any of the accepted modes of administration or agents known in the art. The compositions and/or agents can be administered, for example, parenterally (such as intravenous). The dosage form can be, for example, a solid, semi-solid, lyophilized powder, or liquid dosage form, such as tablets, pills, soft elastic or hard gelatin capsules, powders, solutions, suspensions, suppositories, aerosols, or the like, preferably in unit dosage forms suitable for simple administration of precise dosages.

As discussed above, the mTOR inhibitor nanoparticle composition (such as sirolimus/albumin nanoparticle composition) and a multi-tyrosine kinase inhibitor (e.g., pazopanib) can be administered in a single unit dose or separate dosage forms. Accordingly, the phrase “pharmaceutical combination” includes a combination of two drugs in either a single dosage form or a separate dosage forms, i.e., the pharmaceutically acceptable carriers and excipients described throughout the application can be combined with an mTOR inhibitor nanoparticle composition (such as sirolimus/albumin nanoparticle composition) and a multi-tyrosine kinase inhibitor (e.g., pazopanib) in a single unit dose, as well as individually combined with an mTOR inhibitor nanoparticle composition and a multi-tyrosine kinase inhibitor (e.g., pazopanib) when these compounds are administered separately.

Auxiliary and adjuvant agents may include, for example, preserving, wetting, suspending, sweetening, flavoring, perfuming, emulsifying, and dispensing agents. Prevention of the action of microorganisms is generally provided by various antibacterial and antifungal agents, such as, parabens, chlorobutanol, phenol, sorbic acid, and the like. Isotonic agents, such as sugars, sodium chloride, and the like, may also be included. Prolonged absorption of an injectable pharmaceutical form can be brought about by the use of agents delaying absorption, for example, aluminum monostearate and gelatin. The auxiliary agents also can include wetting agents, emulsifying agents, pH buffering agents, and antioxidants, such as citric acid, sorbitan monolaurate, triethanolamine oleate, butylated hydroxytoluene, and the like.

Solid dosage forms can be prepared with coatings and shells, such as enteric coatings and others well-known in the art. They can contain pacifying agents and can be of such composition that they release the active compound or compounds in a certain part of the intestinal tract in a delayed manner. Examples of embedded compositions that can be used are polymeric substances and waxes. The active compounds also can be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, or dispersing, the mTOR inhibitor nanoparticle composition (such as sirolimus/albumin nanoparticle composition) or multi-tyrosine kinase inhibitor (e.g., pazopanib) described herein, or a pharmaceutically acceptable salt thereof, and optional pharmaceutical adjuvants in a carrier, such as, for example, water, saline, aqueous dextrose, glycerol, ethanol and the like; solubilizing agents and emulsifiers, such as ethyl alcohol, isopropyl alcohol, ethyl carbonate, ethyl acetate, benzyl alcohol, benzyl benzoate, propyleneglycol, 1,3-butyleneglycol, dimethyl formamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

In some embodiments, depending on the intended mode of administration, the pharmaceutically acceptable compositions will contain about 1% to about 99% by weight of the compounds described herein, or a pharmaceutically acceptable salt thereof, and 99% to 1% by weight of a pharmaceutically acceptable excipient. In one example, the composition will be between about 5% and about 75% by weight of a compound described herein, or a pharmaceutically acceptable salt thereof, with the rest being suitable pharmaceutical excipients.

Actual methods of preparing such dosage forms are known, or will be apparent, to those skilled in this art. Reference is made, for example, to Remington's Pharmaceutical Sciences, 18th Ed., (Mack Publishing Company, Easton, Pa., 1990).

The mTOR inhibitor nanoparticle composition (such as sirolimus/albumin nanoparticle composition) can be administered to an individual (such as a human) via various routes, including, for example, via intravenous administration. In some embodiments, sustained continuous release formulation of the composition may be used. In some embodiments, the composition is administered intravenously.

The multi-tyrosine kinase inhibitor (e.g., pazopanib) can be administered to an individual (such as a human) via various routes, including, for example, oral, intravenous, intra-arterial, intraperitoneal, intrapulmonary, inhalation, intravesicular, intramuscular, intra-tracheal, subcutaneous, intraocular, intrathecal, transmucosal, and transdermal. In some embodiments, the multi-tyrosine kinase inhibitor (e.g., pazopanib) is administered orally.

### B. Treatment of Soft-Tissue Sarcoma and Subtypes Thereof.

In some embodiments, there is provided a method of treating a soft-tissue sarcoma in an individual (such as a human) comprising administering to the individual: (a) a composition comprising nanoparticles comprising an mTOR inhibitor (such as a limus drug, e.g., sirolimus or a derivative thereof) and an albumin; and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and (b) a multi-tyrosine kinase inhibitor (e.g., pazopanib). In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the multi-tyrosine kinase inhibitor is pazopanib (e.g., pazopanib hydrochloride). In some embodiments, the mTOR inhibitor nanoparticle composition is administered once out of every 3 weeks, such as on day 1, 8, or 15 of a 21-day cycle. In some embodiments, the mTOR inhibitor nanoparticle composition is administered twice out of every 3 weeks, such as on days 1 and 8 of a 21-day cycle. In some embodiments, the amount of the mTOR inhibitor in the mTOR inhibitor nanoparticle composition is about 10 mg/m² to about 150 mg/m², such as about 1 mg/m² to about 60 mg/m², including about 30 mg/m². In some embodiments, the mTOR inhibitor nanoparticle composition is administered intravenously. In some embodiments, the mTOR inhibitor nanoparticle composition is intravenously administered once out of every 3 weeks, such as on day 1 of a 21-day cycle at about 1 mg/m² to about 60 mg/m², including about 30 mg/m². In some embodiments, the multi-tyrosine kinase inhibitor (e.g., pazopanib) is administered daily. In some embodiments, the amounts of the multi-tyrosine kinase inhibitor (e.g., pazopanib) is about 10 mg to about 800 mg, such as about 400 mg. In some embodiments, the multi-tyrosine kinase inhibitor (e.g., pazopanib) is administered orally. In some embodiments, the multi-tyrosine kinase inhibitor is orally administered daily at about 10 mg to about 800 mg, including about 400 mg. In some embodiments, there is provided a method of treating a soft-tissue sarcoma in an individual (such as a human) comprising: (a) intravenously administering to the individual a composition comprising nanoparticles comprising an mTOR inhibitor (such as a limus drug, e.g., sirolimus or a derivative thereof) and an albumin every three weeks (e.g., on day 1 of a 21-day cycle) at an amount of about 10 mg/m² to about 60 mg/m², such as about 30 mg/m²; and (b) orally administering to the individual pazopanib daily at an amount of about 200 mg to about 600 mg, such as about 400 mg.

In some embodiments, provided is a method of treating a soft-tissue sarcoma in an individual in need thereof, wherein the soft-tissue sarcoma is selected from the group consisting of spindle cell sarcoma, solitary fibrous tumor, leiomyosarcoma, myxofibrosarcoma, undifferentiated pleomorphic sarcoma, melanocytic schwannoma, and pleomorphic rhabdomyosarcoma, the method comprising administering to the individual: (a) a composition comprising nanoparticles comprising an mTOR inhibitor and an albumin; and (b) a multi-tyrosine kinase inhibitor.

Spindle cell sarcoma is an undifferentiated soft-tissue sarcoma originating in the bone, and can present in most parts of the human body, with common locations including the arms, legs, and pelvis. Spindle cell sarcoma is a malignant tumor composed of cells characterized by a long and narrow, or “spindle-cell,” shape and can be categorized as pleomorphic undifferentiated sarcoma, fibrosarcoma, angiosarcoma, and leiomyosarcoma. Diagnosis can be performed by one or more of X-ray, ultrasound, magnetic resonance imaging, and histology from a tumor biopsy. Stradling, “Spindle Cell Sarcoma: Information for Patients,” 2015. Diagnosis of spindle cell sarcoma may be performed by “diagnosis of exclusion” as the tumor has no identifiable characteristics, other than the shape of its cells. A diagnosis of spindle cell sarcoma may be based on its long and narrow cellular morphology, which differs from the most common type of undifferentiated sarcoma, known as undifferentiated pleomorphic sarcoma, which is often comprised of a mix of cells with different sizes and shapes. Sarcoma UK, “What is Sarcoma: Types of Sarcoma: Spindle Cell Sarcoma, 2023; accessible at <https://sarcoma.org.uk/about-sarcoma/what-is-sarcoma/types-of-sarcoma/spindle-cell-sarcoma/>.

In some embodiments, there is provided a method of treating spindle cell sarcoma in an individual (such as a human) comprising administering to the individual: (a) a composition comprising nanoparticles comprising an mTOR inhibitor (such as a limus drug, e.g., sirolimus or a derivative thereof) and an albumin; and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and (b) a multi-tyrosine kinase inhibitor (e.g., pazopanib). In some embodiments, the method comprises administering to the individual: (a a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and (b) a multi-tyrosine kinase inhibitor (e.g., pazopanib). In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the multi-tyrosine kinase inhibitor is pazopanib (e.g., pazopanib hydrochloride). In some embodiments, the mTOR inhibitor nanoparticle composition is administered once out of every 3 weeks, such as on day 1, 8, or 15 of a 21-day cycle. In some embodiments, the mTOR inhibitor nanoparticle composition is administered twice out of every 3 weeks, such as on days 1 and 8 of a 21-day cycle. In some embodiments, the amount of the mTOR inhibitor in the mTOR inhibitor nanoparticle composition is about 10 mg/m² to about 150 mg/m², such as about 1 mg/m² to about 60 mg/m², including about 30 mg/m². In some embodiments, the mTOR inhibitor nanoparticle composition is administered intravenously. In some embodiments, the mTOR inhibitor nanoparticle composition is intravenously administered once out of every 3 weeks, such as on day 1 of a 21-day cycle at about 1 mg/m² to about 60 mg/m², including about 30 mg/m². In some embodiments, the multi-tyrosine kinase inhibitor (e.g., pazopanib) is administered daily. In some embodiments, the amounts of the multi-tyrosine kinase inhibitor (e.g., pazopanib) is about 10 mg to about 800 mg, such as about 400 mg. In some embodiments, the multi-tyrosine kinase inhibitor (e.g., pazopanib) is administered orally. In some embodiments, the multi-tyrosine kinase inhibitor is orally administered daily at about 10 mg to about 800 mg, including about 400 mg. In some embodiments, there is provided a method of treating spindle cell sarcoma in an individual (such as a human) comprising: (a) intravenously administering to the individual a composition comprising nanoparticles comprising an mTOR inhibitor (such as a limus drug, e.g., sirolimus or a derivative thereof) and an albumin every three weeks (e.g., on day 1 of a 21-day cycle) at an amount of about 10 mg/m² to about 60 mg/m², such as about 30 mg/m²; and (b) orally administering to the individual pazopanib daily at an amount of about 200 mg to about 600 mg, such as about 400 mg.

Solitary fibrous tumor is a subtype of soft-tissue sarcoma that can be found in most parts of the human body, with common presentations including intra-thoracic and intra-abdominal locations. Spindle cell sarcoma is a malignant mesenchymal tumor comprised of cells with oval to spindle-shaped nuclei with minimal cytoplasm and intervening collagen bands arranged in patternless distribution with areas highly rich in tumor cells while other areas are more hypocellular with higher percentage of stromal collagen. With gross and histologic features that overlap with many other soft tissue tumors, solitary fibrous tumors have historically been assigned many different names including for example benign mesothelioma, localized mesothelioma, solitary fibrous mesothelioma, and localized fibrous tumor. Like many other soft tissue masses, solitary fibrous tumors are often diagnosed incidentally on imaging. Plain chest radiography can show a well-defined mass of variable size originating from the pleura with or without a pedicle. Contrast enhanced computed tomography can demonstrate a well-circumscribed, often lobulated, hypervascular tumor often with areas of necrosis especially when large. T2-weighted magnetic resonance imaging can reveal a well-defined mass that is markedly inhomogeneous with large areas of bright signaling reflecting extensive areas of necrosis. Furthermore, solitary fibrous tumor can be diagnosed via histopathology of tumor samples, such as from a core needle technique or biopsy. Molecular markers can be used to diagnose solitary fibrous tumor and include a NAB2-STAT6 gene fusion product. However, differential of a soft tissue mass with STAT6 expression may also include dedifferentiated liposarcoma, and further analysis and confirmation of positivity for MDM2 and CKD4 may help differentiate the two and confirm a diagnosis of dedifferentiated liposarcoma rather than solitary fibrous tumor. Davanzo, et al., Transl Gastroenterol Hepatol, 3, 2018.

In some embodiments, there is provided a method of treating solitary fibrous tumor in an individual (such as a human) comprising administering to the individual: (a) a composition comprising nanoparticles comprising an mTOR inhibitor (such as a limus drug, e.g., sirolimus or a derivative thereof) and an albumin; and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and (b) a multi-tyrosine kinase inhibitor (e.g., pazopanib). In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the multi-tyrosine kinase inhibitor is pazopanib (e.g., pazopanib hydrochloride). In some embodiments, the mTOR inhibitor nanoparticle composition is administered once out of every 3 weeks, such as on day 1, 8, or 15 of a 21-day cycle. In some embodiments, the mTOR inhibitor nanoparticle composition is administered twice out of every 3 weeks, such as on days 1 and 8 of a 21-day cycle. In some embodiments, the amount of the mTOR inhibitor in the mTOR inhibitor nanoparticle composition is about 10 mg/m² to about 150 mg/m², such as about 1 mg/m² to about 60 mg/m², including about 30 mg/m². In some embodiments, the mTOR inhibitor nanoparticle composition is administered intravenously. In some embodiments, the mTOR inhibitor nanoparticle composition is intravenously administered once out of every 3 weeks, such as on day 1 of a 21-day cycle at about 1 mg/m² to about 60 mg/m², including about 30 mg/m². In some embodiments, the multi-tyrosine kinase inhibitor (e.g., pazopanib) is administered daily. In some embodiments, the amounts of the multi-tyrosine kinase inhibitor (e.g., pazopanib) is about 10 mg to about 800 mg, such as about 400 mg. In some embodiments, the multi-tyrosine kinase inhibitor (e.g., pazopanib) is administered orally. In some embodiments, the multi-tyrosine kinase inhibitor is orally administered daily at about 10 mg to about 800 mg, including about 400 mg. In some embodiments, there is provided a method of treating solitary fibrous tumor in an individual (such as a human) comprising: (a) intravenously administering to the individual a composition comprising nanoparticles comprising an mTOR inhibitor (such as a limus drug, e.g., sirolimus or a derivative thereof) and an albumin every three weeks (e.g., on day 1 of a 21-day cycle) at an amount of about 10 mg/m² to about 60 mg/m², such as about 30 mg/m²; and (b) orally administering to the individual pazopanib daily at an amount of about 200 mg to about 600 mg, such as about 400 mg.

Leiomyosarcoma is a subtype of soft tissue sarcoma, presented in most parts of the human body, with common locations including the abdomen, retroperitoneum, larger blood vessels, and the uterus. Leiomyosarcoma is a malignant mesenchymal tumor composed of cells that show distinct features of the smooth muscle lineage, and can be categorized as somatic soft tissue leiomyosarcoma, cutaneous leiomyosarcoma, or vascular leiomyosarcoma. Diagnosis can be performed on a tumor sample, such as obtained from a core needle biopsy. Leiomyosarcoma exhibit areas of high cellularity, commonly arranged in fascicles, and malignant cells are characterized by abundant pink to deep red cytoplasm on hematoxylin and eosin (H&E) staining, with cigar-shaped, centrally located nuclei. Such distinguishing features are lost in dedifferentiated tumors. EL-Naggar et al., Cancer Genomics, Chapter 22, 2014. In some embodiments, the leiomyosarcoma is uterine leiomyosarcoma. In some embodiments, the leiomyosarcoma is non-uterine leiomyosarcoma.

In some embodiments, there is provided a method of treating leiomyosarcoma (such as uterine or non-uterine leiomyosarcoma) in an individual (such as a human) comprising administering to the individual: (a) a composition comprising nanoparticles comprising an mTOR inhibitor (such as a limus drug, e.g., sirolimus or a derivative thereof) and an albumin; and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and (b) a multi-tyrosine kinase inhibitor (e.g., pazopanib). In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the multi-tyrosine kinase inhibitor is pazopanib (e.g., pazopanib hydrochloride). In some embodiments, the mTOR inhibitor nanoparticle composition is administered once out of every 3 weeks, such as on day 1, 8, or 15 of a 21-day cycle. In some embodiments, the mTOR inhibitor nanoparticle composition is administered twice out of every 3 weeks, such as on days 1 and 8 of a 21-day cycle. In some embodiments, the amount of the mTOR inhibitor in the mTOR inhibitor nanoparticle composition is about 10 mg/m² to about 150 mg/m², such as about 1 mg/m² to about 60 mg/m², including about 30 mg/m². In some embodiments, the mTOR inhibitor nanoparticle composition is administered intravenously. In some embodiments, the mTOR inhibitor nanoparticle composition is intravenously administered once out of every 3 weeks, such as on day 1 of a 21-day cycle at about 1 mg/m² to about 60 mg/m², including about 30 mg/m². In some embodiments, the multi-tyrosine kinase inhibitor (e.g., pazopanib) is administered daily. In some embodiments, the amounts of the multi-tyrosine kinase inhibitor (e.g., pazopanib) is about 10 mg to about 800 mg, such as about 400 mg. In some embodiments, the multi-tyrosine kinase inhibitor (e.g., pazopanib) is administered orally. In some embodiments, the multi-tyrosine kinase inhibitor is orally administered daily at about 10 mg to about 800 mg, including about 400 mg. In some embodiments, there is provided a method of treating leiomyosarcoma in an individual (such as a human) comprising: (a) intravenously administering to the individual a composition comprising nanoparticles comprising an mTOR inhibitor (such as a limus drug, e.g., sirolimus or a derivative thereof) and an albumin every three weeks (e.g., on day 1 of a 21-day cycle) at an amount of about 10 mg/m² to about 60 mg/m², such as about 30 mg/m²; and (b) orally administering to the individual pazopanib daily at an amount of about 200 mg to about 600 mg, such as about 400 mg.

Myxofibrosarcoma is an aggressive soft-tissue sarcoma commonly found in the extremities of elderly patients. It is classified as a type of a fibroblastic/myofibroblastic tumor. Clinically, the primary lesion may present as a predominantly deep or subcutaneous multinodular growth. It also may involve the dermal layer and present as a cutaneous lesion. Grossly, myxofibrosarcoma consists of nodules ranging in size from a few millimeters to 1-2 cm, usually less than 5 cm. Tumor nodules are separated by thin fibrous septa and a rich arborizing vasculature. The tumor is soft with a gray-white mucoid appearance and areas of necrosis and hemorrhage. Waters et al., American Journal of Roentgenology, 188: W193-W198, 2007. Myxofibrosarcoma can be diagnosed based on the analysis of cytomorphologic features. The macroscopic appearance of myxofibrosarcoma is characterized by multiple variably gelatinous or firmer nodules in the superficially located lesions, while infiltrative margins are often present in deep-seated diseases. From a molecular point of view, myxofibrosarcomas exhibit highly complex karyotypes with a number of different genomic aberrations. At present no specific molecular pathology markers are available for the standard differential diagnosis. Vanni et al., Ther Adv Med Oncol, 14, 2002

In some embodiments, there is provided a method of treating myxofibrosarcoma in an individual (such as a human) comprising administering to the individual: (a) a composition comprising nanoparticles comprising an mTOR inhibitor (such as a limus drug, e.g., sirolimus or a derivative thereof) and an albumin; and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and (b) a multi-tyrosine kinase inhibitor (e.g., pazopanib). In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the multi-tyrosine kinase inhibitor is pazopanib (e.g., pazopanib hydrochloride). In some embodiments, the mTOR inhibitor nanoparticle composition is administered once out of every 3 weeks, such as on day 1, 8, or 15 of a 21-day cycle. In some embodiments, the mTOR inhibitor nanoparticle composition is administered twice out of every 3 weeks, such as on days 1 and 8 of a 21-day cycle. In some embodiments, the amount of the mTOR inhibitor in the mTOR inhibitor nanoparticle composition is about 10 mg/m² to about 150 mg/m², such as about 1 mg/m² to about 60 mg/m², including about 30 mg/m². In some embodiments, the mTOR inhibitor nanoparticle composition is administered intravenously. In some embodiments, the mTOR inhibitor nanoparticle composition is intravenously administered once out of every 3 weeks, such as on day 1 of a 21-day cycle at about 1 mg/m² to about 60 mg/m², including about 30 mg/m². In some embodiments, the multi-tyrosine kinase inhibitor (e.g., pazopanib) is administered daily. In some embodiments, the amounts of the multi-tyrosine kinase inhibitor (e.g., pazopanib) is about 10 mg to about 800 mg, such as about 400 mg. In some embodiments, the multi-tyrosine kinase inhibitor (e.g., pazopanib) is administered orally. In some embodiments, the multi-tyrosine kinase inhibitor is orally administered daily at about 10 mg to about 800 mg, including about 400 mg. In some embodiments, there is provided a method of treating myxofibrosarcoma in an individual (such as a human) comprising: (a) intravenously administering to the individual a composition comprising nanoparticles comprising an mTOR inhibitor (such as a limus drug, e.g., sirolimus or a derivative thereof) and an albumin every three weeks (e.g., on day 1 of a 21-day cycle) at an amount of about 10 mg/m² to about 60 mg/m², such as about 30 mg/m²; and (b) orally administering to the individual pazopanib daily at an amount of about 200 mg to about 600 mg, such as about 400 mg.

Undifferentiated pleomorphic sarcoma (UPS) is a high-grade, and often aggressive, soft-tissue sarcoma. Undifferentiated pleomorphic sarcoma usually appear as asymptomatic, unremarkable, rapidly growing cutaneous or subcutaneous nodule without superficial skin abnormalities. It is likely that the origin of undifferentiated pleomorphic sarcoma is mesenchymal stem cells. Undifferentiated pleomorphic sarcoma has been found to affect, amongst other areas, bones, soft tissues, and the retroperitoneum, and can also metastasize to other organs. Undifferentiated pleomorphic sarcoma can be diagnosed via histopathology of tumor samples, such as from a core needle technique or biopsy. Markers can be used to diagnose undifferentiated pleomorphic sarcoma, and include keratins, S100 protein, and/or SOX10, smooth muscle actin (SMA), and desmin. MDM2 and CDK4 may also be helpful to distinguish undifferentiated pleomorphic sarcoma from dedifferentiated liposarcoma. Undifferentiated pleomorphic sarcoma exhibits atypical, pleomorphic spindle cells with abundant mitotic figures, and the tumor may display storiform, fascicular, or sheet-like configuration within a fibrous stroma. Robles-Tenorio & Solis-Ledesma, StatPearls, Undifferentiated Pleomorphic Sarcoma, 2022.

In some embodiments, there is provided a method of treating undifferentiated pleomorphic sarcoma in an individual (such as a human) comprising administering to the individual: (a) a composition comprising nanoparticles comprising an mTOR inhibitor (such as a limus drug, e.g., sirolimus or a derivative thereof) and an albumin; and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and (b) a multi-tyrosine kinase inhibitor (e.g., pazopanib). In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the multi-tyrosine kinase inhibitor is pazopanib (e.g., pazopanib hydrochloride). In some embodiments, the mTOR inhibitor nanoparticle composition is administered once out of every 3 weeks, such as on day 1, 8, or 15 of a 21-day cycle. In some embodiments, the mTOR inhibitor nanoparticle composition is administered twice out of every 3 weeks, such as on days 1 and 8 of a 21-day cycle. In some embodiments, the amount of the mTOR inhibitor in the mTOR inhibitor nanoparticle composition is about 10 mg/m² to about 150 mg/m², such as about 1 mg/m² to about 60 mg/m², including about 30 mg/m². In some embodiments, the mTOR inhibitor nanoparticle composition is administered intravenously. In some embodiments, the mTOR inhibitor nanoparticle composition is intravenously administered once out of every 3 weeks, such as on day 1 of a 21-day cycle at about 1 mg/m² to about 60 mg/m², including about 30 mg/m². In some embodiments, the multi-tyrosine kinase inhibitor (e.g., pazopanib) is administered daily. In some embodiments, the amounts of the multi-tyrosine kinase inhibitor (e.g., pazopanib) is about 10 mg to about 800 mg, such as about 400 mg. In some embodiments, the multi-tyrosine kinase inhibitor (e.g., pazopanib) is administered orally. In some embodiments, the multi-tyrosine kinase inhibitor is orally administered daily at about 10 mg to about 800 mg, including about 400 mg. In some embodiments, there is provided a method of treating undifferentiated pleomorphic sarcoma in an individual (such as a human) comprising: (a) intravenously administering to the individual a composition comprising nanoparticles comprising an mTOR inhibitor (such as a limus drug, e.g., sirolimus or a derivative thereof) and an albumin every three weeks (e.g., on day 1 of a 21-day cycle) at an amount of about 10 mg/m² to about 60 mg/m², such as about 30 mg/m²; and (b) orally administering to the individual pazopanib daily at an amount of about 200 mg to about 600 mg, such as about 400 mg.

Melanocytic schwannoma is a rare soft-tissue sarcoma that arises most commonly in the paraspinal sympathetic chain. Histologically, melanotic schwannomas are characterized by the typical spindle shaped neoplastic Schwann cells and melanosomes in various stages of maturation. These tumors are grossly pigmented and immunophenotypically mark for both Schwann cell markers and markers of melanocytic differentiation. Scheithauer, et al., WHO Classification of Tumours of the Central Nervous System, pp. 152-5, 2007. Melanocytic schwannoma can be diagnosed based on imaging methods. When originating from spinal nerves, melanocytic schwannoma can be diagnosed by radiographs and computed tomography scans which can show enlargement of the intervertebral foramina, bone erosion, and sclerosis, with a “dumbbell” morphology. Further, myelograms can depict obstruction of the contrast flow without displacement of the spinal cord, and tumors contacting bone can show cortical erosion, sclerosis, and local destruction. Microscopically, melanocytic schwannomas are circumscribed but not encapsulated and contain plump spindle and epithelioid cells arranged in interlacing fascicles or nests. Melanocytic schwannoma tumors may be characterized by accumulation of melanin in neoplastic cells and associated melanophages. Alexiev, et al., Arch Pathol Lab Med (2018) 142 (12): 1517-1523.

In some embodiments, there is provided a method of treating melanocytic schwannoma in an individual (such as a human) comprising administering to the individual: (a) a composition comprising nanoparticles comprising an mTOR inhibitor (such as a limus drug, e.g., sirolimus or a derivative thereof) and an albumin; and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and (b) a multi-tyrosine kinase inhibitor (e.g., pazopanib). In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the multi-tyrosine kinase inhibitor is pazopanib (e.g., pazopanib hydrochloride). In some embodiments, the mTOR inhibitor nanoparticle composition is administered once out of every 3 weeks, such as on day 1, 8, or 15 of a 21-day cycle. In some embodiments, the mTOR inhibitor nanoparticle composition is administered twice out of every 3 weeks, such as on days 1 and 8 of a 21-day cycle. In some embodiments, the amount of the mTOR inhibitor in the mTOR inhibitor nanoparticle composition is about 10 mg/m² to about 150 mg/m², such as about 1 mg/m² to about 60 mg/m², including about 30 mg/m². In some embodiments, the mTOR inhibitor nanoparticle composition is administered intravenously. In some embodiments, the mTOR inhibitor nanoparticle composition is intravenously administered once out of every 3 weeks, such as on day 1 of a 21-day cycle at about 1 mg/m² to about 60 mg/m², including about 30 mg/m². In some embodiments, the multi-tyrosine kinase inhibitor (e.g., pazopanib) is administered daily. In some embodiments, the amounts of the multi-tyrosine kinase inhibitor (e.g., pazopanib) is about 10 mg to about 800 mg, such as about 400 mg. In some embodiments, the multi-tyrosine kinase inhibitor (e.g., pazopanib) is administered orally. In some embodiments, the multi-tyrosine kinase inhibitor is orally administered daily at about 10 mg to about 800 mg, including about 400 mg. In some embodiments, there is provided a method of treating melanocytic schwannoma in an individual (such as a human) comprising: (a) intravenously administering to the individual a composition comprising nanoparticles comprising an mTOR inhibitor (such as a limus drug, e.g., sirolimus or a derivative thereof) and an albumin every three weeks (e.g., on day 1 of a 21-day cycle) at an amount of about 10 mg/m² to about 60 mg/m², such as about 30 mg/m²; and (b) orally administering to the individual pazopanib daily at an amount of about 200 mg to about 600 mg, such as about 400 mg.

Pleomorphic rhabdomysarcoma is a rare high-grade soft-tissue sarcoma comprised of undifferentiated round and spindle cells that display skeletal-muscle differentiation without embryonal or alveolar components. Typically, it is an aggressive lesion arising in the deep soft tissues of the extremities with a high propensity for metastasis. Pleomorphic rhabdomysarcoma presents in the deep soft tissues most commonly in the extremities, with less common locations including the pelvis, abdomen, thorax and head and neck. Diagnosis of pleomorphic rhabdomysarcoma may be a diagnosis of exclusion, combining morphological features, immunoprofiling and molecular profiling to exclude alternative diagnoses. Noujaim et al., Anticancer Research, 2015; 35 (11)6213-6217. Diagnostic criteria for pleomorphic rhabdomysarcoma remain controversial. Thorough histopathology and immunohistochemistry analyses demonstrating a skeletal muscle phenotype may be used to confirm pleomorphic rhabdomysarcoma diagnosis, in conjunction with immunohistochemical analysis revealing expression of vimentin, desmin, SMA, myogenin, and MyoD1, but not S100 and CD117. Xi et al., Medicine, 97(51):p e13648, 2018.

In some embodiments, there is provided a method of treating pleomorphic rhabdomyosarcoma in an individual (such as a human) comprising administering to the individual: (a) a composition comprising nanoparticles comprising an mTOR inhibitor (such as a limus drug, e.g., sirolimus or a derivative thereof) and an albumin; and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and (b) a multi-tyrosine kinase inhibitor (e.g., pazopanib). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and (b) a multi-tyrosine kinase inhibitor (e.g., pazopanib). In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the multi-tyrosine kinase inhibitor is pazopanib (e.g., pazopanib hydrochloride). In some embodiments, the mTOR inhibitor nanoparticle composition is administered once out of every 3 weeks, such as on day 1, 8, or 15 of a 21-day cycle. In some embodiments, the mTOR inhibitor nanoparticle composition is administered twice out of every 3 weeks, such as on days 1 and 8 of a 21-day cycle. In some embodiments, the amount of the mTOR inhibitor in the mTOR inhibitor nanoparticle composition is about 10 mg/m² to about 150 mg/m², such as about 1 mg/m² to about 60 mg/m², including about 30 mg/m². In some embodiments, the mTOR inhibitor nanoparticle composition is administered intravenously. In some embodiments, the mTOR inhibitor nanoparticle composition is intravenously administered once out of every 3 weeks, such as on day 1 of a 21-day cycle at about 1 mg/m² to about 60 mg/m², including about 30 mg/m². In some embodiments, the multi-tyrosine kinase inhibitor (e.g., pazopanib) is administered daily. In some embodiments, the amounts of the multi-tyrosine kinase inhibitor (e.g., pazopanib) is about 10 mg to about 800 mg, such as about 400 mg. In some embodiments, the multi-tyrosine kinase inhibitor (e.g., pazopanib) is administered orally. In some embodiments, the multi-tyrosine kinase inhibitor is orally administered daily at about 10 mg to about 800 mg, including about 400 mg. In some embodiments, there is provided a method of treating pleomorphic rhabdomyosarcoma in an individual (such as a human) comprising: (a) intravenously administering to the individual a composition comprising nanoparticles comprising an mTOR inhibitor (such as a limus drug, e.g., sirolimus or a derivative thereof) and an albumin every three weeks (e.g., on day 1 of a 21-day cycle) at an amount of about 10 mg/m² to about 60 mg/m², such as about 30 mg/m²; and (b) orally administering to the individual pazopanib daily at an amount of about 200 mg to about 600 mg, such as about 400 mg.

III. Compositions Comprising Nanoparticles Comprising an mTOR Inhibitor

The mTOR inhibitor nanoparticle compositions described herein comprise nanoparticles comprising (in various embodiments consisting essentially of or consisting of) an mTOR inhibitor (such as a limus drug, e.g., rapamycin or a derivative thereof) and an albumin (such as human serum albumin). Nanoparticles of poorly water soluble drugs (such as macrolides) have been disclosed in, for example, U. S. Pat. Nos. 5,916,596; 6,506,405; 6,749,868, 6,537,579, 7,820,788, and 8,911,786, 11,497,737, and also in U. S. Pat. Pub. Nos. 2006/0263434, and 2007/0082838; PCT Patent Application WO08/137148, U.S. Patent Application No. 62/927,047, each of which is incorporated herein by reference in their entirety.

In some embodiments, the composition comprises nanoparticles with an average or mean diameter of no greater than about 1000 nanometers (nm), such as no greater than about any of 900, 800, 700, 600, 500, 400, 300, 200, and 100 nm. In some embodiments, the average or mean diameters of the nanoparticles is no greater than about 200 nm. In some embodiments, the average or mean diameters of the nanoparticles is no greater than about 150 nm. In some embodiments, the average or mean diameters of the nanoparticles is no greater than about 100 nm. In some embodiments, the average or mean diameter of the nanoparticles is about 10 to about 400 nm. In some embodiments, the average or mean diameter of the nanoparticles is about 10 to about 150 nm. In some embodiments, the average or mean diameter of the nanoparticles is about 40 to about 120 nm. In some embodiments, the average or mean diameter of the nanoparticles are no less than about 50 nm. In some embodiments, the nanoparticles are sterile-filterable.

Methods of determining average particle sizes are known in the art, for example, dynamic light scattering (DLS) has been routinely used in determining the size of submicrometre-sized particles based. International Standard ISO22412 Particle Size Analysis-Dynamic Light Scattering, International Organisation for Standardisation (ISO) 2008 and Dynamic Light Scattering Common Terms Defined, Malvern Instruments Limited, 2011. In some embodiments, the particle size is measured as the volume-weighted mean particle size (Dv50) of the nanoparticles in the composition.

In some embodiments, the nanoparticles comprise the mTOR inhibitor associated with the albumin. In some embodiments, the nanoparticles comprise the mTOR inhibitor coated with the albumin.

In some embodiments, the albumin has sulfhydryl groups that can form disulfide bonds. In some embodiments, at least about 5% (including for example at least about any one of 10%, 15%, 20%, 25%, 30%, 40%, 50%, 60%, 70%, 80%, or 90%) of the albumin in the nanoparticle portion of the composition are crosslinked (for example crosslinked through one or more disulfide bonds).

In some embodiments, the nanoparticles comprising the mTOR inhibitor (such as a limus drug, e.g., rapamycin or a derivative thereof) are associated (e.g., coated) with an albumin (such as human albumin or human serum albumin). In some embodiments, the composition comprises an mTOR inhibitor (such as a limus drug, e.g., rapamycin or a derivative thereof) in both nanoparticle and non-nanoparticle forms (e.g., in the form of solutions or in the form of soluble albumin/nanoparticle complexes), wherein at least about any one of 50%, 60%, 70%, 80%, 90%, 95%, or 99% of the mTOR inhibitor in the composition are in nanoparticle form. In some embodiments, the mTOR inhibitor (such as a limus drug, e.g., rapamycin or a derivative thereof) in the nanoparticles constitutes more than about any one of 50%, 60%, 70%, 80%, 90%, 95%, or 99% of the nanoparticles by weight. In some embodiments, the nanoparticles have a non-polymeric matrix. In some embodiments, the nanoparticles comprise a core of an mTOR inhibitor (such as a limus drug, e.g., rapamycin or a derivative thereof) that is substantially free of polymeric materials (such as polymeric matrix).

In some embodiments, the composition comprises an albumin in both nanoparticle and non-nanoparticle portions of the composition, wherein at least about any one of 50%, 60%, 70%, 80%, 90%, 95%, or 99% of the albumin in the composition are in non-nanoparticle portion of the composition.

In some embodiments, the weight ratio of the albumin to the mTOR inhibitor (such as a limus drug, e.g., rapamycin or a derivative thereof) in the mTOR inhibitor nanoparticle composition is such that a sufficient amount of mTOR inhibitor binds to, or is transported by, the cell. While the weight ratio of an albumin to an mTOR inhibitor (such as a limus drug, e.g., rapamycin or a derivative thereof) will have to be optimized for different albumin and mTOR inhibitor combinations, generally the weight ratio of an albumin to an mTOR inhibitor (such as a limus drug, e.g., rapamycin or a derivative thereof) (w/w) is about 0.01:1 to about 100:1, about 0.02:1 to about 50:1, about 0.05:1 to about 20:1, about 0.1:1 to about 20:1, about 1:1 to about 18:1, about 2:1 to about 15:1, about 3:1 to about 12:1, about 4:1 to about 10:1, about 5:1 to about 9:1, or about 9:1. In some embodiments, the albumin to mTOR inhibitor (such as a limus drug, e.g., rapamycin or a derivative thereof) weight ratio is about any of 18:1 or less, 15:1 or less, 14:1 or less, 13:1 or less, 12:1 or less, 11:1 or less, 10:1 or less, 9:1 or less, 8:1 or less, 7:1 or less, 6:1 or less, 5:1 or less, 4:1 or less, and 3:1 or less. In some embodiments, the weight ratio of the albumin (such as human albumin or human serum albumin) to the mTOR inhibitor (such as a limus drug, e.g., rapamycin or a derivative thereof) in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1.

In some embodiments, the composition comprises nanoparticles comprising an mTOR inhibitor and an albumin, wherein the weight ratio of the albumin to the mTOR inhibitor in the composition is about 0.01:1 to about 100:1. In some embodiments, the composition comprises nanoparticles comprising an mTOR inhibitor (such as rapamycin) and an albumin, wherein the weight ratio of the albumin to the mTOR inhibitor (such as rapamycin) in the composition is about 18:1 or less (including for example any of about 1:1 to about 18:1, about 2:1 to about 15:1, about 3:1 to about 12:1, about 4:1 to about 10:1, about 5:1 to about 9:1, and about 9:1). In some embodiments, the composition comprises nanoparticles comprising rapamycin, or a derivative thereof, and an albumin, wherein the weight ratio of the albumin to the rapamycin or derivative thereof in the composition is about 18:1 or less (including for example any of about 1:1 to about 18:1, about 2:1 to about 15:1, about 3:1 to about 12:1, about 4:1 to about 10:1, about 5:1 to about 9:1, and about 9:1). In some embodiments, the mTOR inhibitor (such as rapamycin) is coated with albumin.

In some embodiments, the mTOR inhibitor nanoparticle composition (such as rapamycin/albumin nanoparticle composition) comprises one or more of the above characteristics.

The nanoparticles described herein may be present in a dry formulation (such as lyophilized composition) or suspended in a biocompatible medium. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

In some embodiments, the pharmaceutically acceptable carrier comprises an albumin (such as human albumin or human serum albumin). The albumin may either be natural in origin or synthetically prepared. In some embodiments, the albumin is human albumin or human serum albumin. In some embodiments, the albumin is a recombinant albumin.

Human serum albumin (HSA) is a highly soluble globular protein of Mᵣ 65K and consists of 585 amino acids. HSA is the most abundant protein in the plasma and accounts for 70-80% of the colloid osmotic pressure of human plasma. The amino acid sequence of HSA contains a total of 17 disulfide bridges, one free thiol (Cys 34), and a single tryptophan (Trp 214). Intravenous use of HSA solution has been indicated for the prevention and treatment of hypovolemic shock (see, e.g., Tullis, JAMA, 237:355-360, 460-463, (1977)) and Houser et al., Surgery, Gynecology and Obstetrics, 150:811-816 (1980)) and in conjunction with exchange transfusion in the treatment of neonatal hyperbilirubinemia (see, e.g., Finlayson, Seminars in Thrombosis and Hemostasis, 6, 85-120, (1980)). Other albumins are contemplated, such as bovine serum albumin. Use of such non-human albumins could be appropriate, for example, in the context of use of these compositions in non-human mammals, such as the veterinary (including domestic pets and agricultural context). Human serum albumin (HSA) has multiple hydrophobic binding sites (a total of eight for fatty acids, an endogenous ligand of HSA) and binds a diverse set of drugs, especially neutral and negatively charged hydrophobic compounds (Goodman et al., The Pharmacological Basis of Therapeutics, 9^{th} ed, McGraw-Hill New York (1996)). Two high affinity binding sites have been proposed in subdomains IIA and IIIA of HSA, which are highly elongated hydrophobic pockets with charged lysine and arginine residues near the surface which function as attachment points for polar ligand features (see, e.g., Fehske et al., Biochem. Pharmcol., 30, 687-92 (198a), Vorum, Dan. Med. Bull., 46, 379-99 (1999), Kragh-Hansen, Dan. Med. Bull., 1441, 131-40 (1990), Curry et al., Nat. Struct. Biol., 5, 827-35 (1998), Sugio et al., Protein. Eng., 12, 439-46 (1999), He et al., Nature, 358, 209-15 (199b), and Carter et al., Adv. Protein. Chem., 45, 153-203 (1994)). Rapamycin and propofol have been shown to bind HSA (see, e.g., Paal et al., Eur. J. Biochem., 268 (7), 2187-91 (200a), Purcell et al., Biochem. Biophys. Acta, 1478 (a), 61-8 (2000), Altmayer et al., Arzneimittelforschung, 45, 1053-6 (1995), and Garrido et al., Rev. Esp. Anestestiol. Reanim., 41, 308-12 (1994)). In addition, docetaxel has been shown to bind to human plasma proteins (see, e.g., Urien et al., Invest. New Drugs, 14(b), 147-51 (1996)).

An mTOR inhibitor (such as a limus drug, e.g., rapamycin or a derivative thereof) is “stabilized” in an aqueous suspension if it remains suspended in an aqueous medium (such as without visible precipitation or sedimentation) for an extended period of time, such as for at least about any of 0.1, 0.2, 0.25, 0.5, 1, 2, 3, 4, 5, 6, 7, 8, 9, 10, 11, 12, 24, 36, 48, 60, or 72 hours. The suspension is generally, but not necessarily, suitable for administration to an individual (such as a human). Stability of the suspension is generally (but not necessarily) evaluated at a storage temperature (such as room temperature (such as 20-25° C.) or refrigerated conditions (such as 4° C.)). For example, a suspension is stable at a storage temperature if it exhibits no flocculation or particle agglomeration visible to the naked eye or when viewed using an optical microscope at 1000 times, at about fifteen minutes after preparation of the suspension. Stability can also be evaluated under accelerated testing conditions, such as at a temperature that is about 40° C. or higher.

The compositions described herein may be a stable aqueous suspension of the mTOR inhibitor, such as a stable aqueous suspension of the mTOR inhibitor at a concentration of any of about 0.1 to about 200 mg/ml, about 0.1 to about 150 mg/ml, about 0.1 to about 100 mg/ml, about 0.1 to about 50 mg/ml, about 0.1 to about 20 mg/ml, about 1 to about 10 mg/ml, about 2 mg/ml to about 8 mg/ml, about 4 to about 6 mg/ml, and about 5 mg/ml. In some embodiments, the concentration of the mTOR inhibitor is at least about any of 0.2 mg/ml, 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, 50 mg/ml, 100 mg/ml, 150 mg/ml, or 200 mg/ml.

In some embodiments, the albumin is present in an amount that is sufficient to stabilize the mTOR inhibitor (such as a limus drug, e.g., rapamycin or a derivative thereof) in an aqueous suspension at a certain concentration. For example, the concentration of the mTOR inhibitor (such as a limus drug, e.g., rapamycin or a derivative thereof) in the composition is about 0.1 to about 100 mg/ml, including for example about any of 0.1 to about 50 mg/ml, about 0.1 to about 20 mg/ml, about 1 to about 10 mg/ml, about 2 mg/ml to about 8 mg/ml, about 4 to about 6 mg/ml, or about 5 mg/ml. In some embodiments, the concentration of the mTOR inhibitor (such as a limus drug, e.g., rapamycin or a derivative thereof) is at least about any of 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, and 50 mg/ml. In some embodiments, the albumin is present in an amount that avoids use of surfactants (such as Cremophor), so that the composition is free or substantially free of surfactant (such as Cremophor).

In some embodiments, the composition, in liquid form, comprises from about 0.1% to about 50% (w/v) (e.g., about 0.5% (w/v), about 5% (w/v), about 10% (w/v), about 15% (w/v), about 20% (w/v), about 30% (w/v), about 40% (w/v), or about 50% (w/v)) of an albumin. In some embodiments, the composition, in liquid form, comprises about 0.5% to about 5% (w/v) of albumin.

In some embodiments, the albumin allows the composition to be administered to an individual (such as a human) without significant side effects. In some embodiments, the albumin (such as human serum albumin or human albumin) is in an amount that is effective to reduce one or more side effects of administration of the mTOR inhibitor (such as a limus drug, e.g., rapamycin or a derivative thereof) to a human. The term “reducing one or more side effects” of administration of the mTOR inhibitor (such as a limus drug, e.g., rapamycin or a derivative thereof) refers to reduction, alleviation, elimination, or avoidance of one or more undesirable effects caused by the mTOR inhibitor, as well as side effects caused by delivery vehicles (such as solvents that render the limus drugs suitable for injection) used to deliver the mTOR inhibitor. Such side effects include, for example, myelosuppression, neurotoxicity, hypersensitivity, inflammation, venous irritation, phlebitis, pain, skin irritation, peripheral neuropathy, neutropenic fever, anaphylactic reaction, venous thrombosis, extravasation, and combinations thereof. These side effects, however, are merely exemplary and other side effects, or combination of side effects, associated with limus drugs (such as a limus drug, e.g., rapamycin or a derivative thereof) can be reduced.

In some embodiments, the composition is a dry (such as lyophilized) composition that can be reconstituted, resuspended, or rehydrated to form generally a stable aqueous suspension of the nanoparticles comprising an mTOR inhibitor and an albumin. In some embodiments, the composition is a liquid (such as aqueous) composition obtained by reconstituting or resuspending a dry composition. In some embodiments, the composition is an intermediate liquid (such as aqueous) composition that can be dried (such as lyophilized).

A. mTOR Inhibitors

The methods described herein in some embodiments comprise administration of nanoparticle compositions of mTOR inhibitors. “mTOR inhibitor” used herein refers to an inhibitor of mTOR. mTOR is a serine/threonine-specific protein kinase downstream of the phosphatidylinositol 3-kinase (PI3K)/Akt (protein kinase B) pathway, and a key regulator of cell survival, proliferation, stress, and metabolism. mTOR pathway dysregulation has been found in many human carcinomas, and mTOR inhibition produced substantial inhibitory effects on tumor progression.

The mammalian target of rapamycin (mTOR) (also known as mechanistic target of rapamycin or FK506 binding protein 12-rapamycin associated protein 1 (FRAP1)) is an atypical serine/threonine protein kinase that is present in two distinct complexes, mTOR Complex 1 (mTORC1) and mTOR Complex 2 (mTORC2). mTORC1 is composed of mTOR, regulatory-associated protein of mTOR (Raptor), mammalian lethal with SEC13 protein 8 (MLST8), PRAS40 and DEPTOR (Kim et al. (2002). Cell 110:163-75; Fang et al. (2001). Science 294 (5548): 1942-5). mTORC1 integrates four major signal inputs: nutrients (such as amino acids and phosphatidic acid), growth factors (insulin), energy and stress (such as hypoxia and DNA damage). Amino acid availability is signaled to mTORC1 via a pathway involving the Rag and Ragulator (LAMTOR1-3) Growth factors and hormones (e.g., insulin) signal to mTORC1 via Akt, which inactivates TSC2 to prevent inhibition of mTORC1. Alternatively, low ATP levels lead to the AMPK-dependent activation of TSC2 and phosphorylation of raptor to reduce mTORC1 signaling proteins.

Active mTORC1 has a number of downstream biological effects including translation of mRNA via the phosphorylation of downstream targets (4E-BP1 and p70 S6 Kinase), suppression of autophagy (Atg13, ULK1), ribosome biogenesis, and activation of transcription leading to mitochondrial metabolism or adipogenesis. Accordingly, mTORC1 activity promotes either cellular growth when conditions are favorable or catabolic processes during stress or when conditions are unfavorable.

mTORC2 is composed of mTOR, rapamycin-insensitive companion of mTOR (RICTOR), GBL, and mammalian stress-activated protein kinase interacting protein 1 (mSIN1). In contrast to mTORC1, for which many upstream signals and cellular functions have been defined (see above), relatively little is known about mTORC2 biology. mTORC2 regulates cytoskeletal organization through its stimulation of F-actin stress fibers, paxillin, RhoA, Rac1, Cdc42, and protein kinase C α (PKCα). It had been observed that knocking down mTORC2 components affects actin polymerization and perturbs cell morphology (Jacinto et al. (2004). Nat. Cell Biol. 6, 1122-1128; Sarbassov et al. (2004). Curr. Biol. 14, 1296-1302). This suggests that mTORC2 controls the actin cytoskeleton by promoting protein kinase Cα (PKCα) phosphorylation, phosphorylation of paxillin and its relocalization to focal adhesions, and the GTP loading of RhoA and Rac1. The molecular mechanism by which mTORC2 regulates these processes has not been determined.

In some embodiments, the mTOR inhibitor (such as a limus drug, e.g., sirolimus or a derivative thereof) is an inhibitor of mTORC1. In some embodiments, the mTOR inhibitor (such as a limus drug, e.g., sirolimus or a derivative thereof) is an inhibitor of mTORC2. In some embodiments, the mTOR inhibitor (such as a limus drug, e.g., sirolimus or a derivative thereof) is an inhibitor of both mTORC1 and mTORC2.

In some embodiments, the mTOR inhibitor is a limus drug, which includes sirolimus and its analogs. Examples of limus drugs include, but are not limited to, temsirolimus (CCI-779), everolimus (RAD001), ridaforolimus (AP-23573), deforolimus (MK-8669), zotarolimus (ABT-578), pimecrolimus, and tacrolimus (FK-506). In some embodiments, the limus drug is selected from the group consisting of temsirolimus (CCI-779), everolimus (RAD001), ridaforolimus (AP-23573), deforolimus (MK-8669), zotarolimus (ABT-578), pimecrolimus, and tacrolimus (FK-506). In some embodiments, the mTOR inhibitor is an mTOR kinase inhibitor, such as CC-115 or CC-223.

In some embodiments, the mTOR inhibitor is sirolimus. Sirolimus is macrolide antibiotic that complexes with FKBP-12 and inhibits the mTOR pathway by binding mTORC1.

In some embodiments, the mTOR inhibitor is selected from the group consisting of sirolimus (rapamycin), BEZ235 (NVP-BEZ235), everolimus (also known as RAD001, Zortress, Certican, and Afinitor), AZD8055, temsirolimus (also known as CCI-779 and Torisel), CC-115, CC-223, PI-103, Ku-0063794, INK 128, AZD2014, NVP-BGT226, PF-04691502, CH5132799, GDC-0980 (RG7422), Torin 1, WAY-600, WYE-125132, WYE-687, GSK2126458, PF-05212384 (PKI-587), PP-121, OSI-027, Palomid 529, PP242, XL765, GSK1059615, WYE-354, and ridaforolimus (also known as deforolimus).

BEZ235 (NVP-BEZ235) is an imidazoquilonine derivative that is an mTORC1 catalytic inhibitor (Roper J, et al. PLOS One, 2011, 6 (9), e25132). Everolimus is the 40-O-(2-hydroxyethyl) derivative of sirolimus and binds the cyclophilin FKBP-12, and this complex also mTORC1. AZD8055 is a small molecule that inhibits the phosphorylation of mTORC1 (p70S6K and 4E-BP1). Temsirolimus is a small molecule that forms a complex with the FK506-binding protein and prohibits the activation of mTOR when it resides in the mTORCIcomplex. PI-103 is a small molecule that inhibits the activation of the rapamycin-sensitive (mTORC1) complex (Knight et al. (2006) Cell. 125:733-47). KU-0063794 is a small molecule that inhibits the phosphorylation of mTORC1 at Ser2448 in a dose-dependent and time-dependent manner. INK 128, AZD2014, NVP-BGT226, CH5132799, WYE-687, and are each small molecule inhibitors of mTORC1. PF-04691502 inhibits mTORC1 activity. GDC-0980 is an orally bioavailable small molecule that inhibits Class I PI3 Kinase and TORC1. Torin 1 is a potent small molecule inhibitor of mTOR. WAY-600 is a potent, ATP-competitive and selective inhibitor of mTOR. WYE-125132 is an ATP-competitive small molecule inhibitor of mTORC1. GSK2126458 is an inhibitor of mTORC1. PKI-587 is a highly potent dual inhibitor of PI3Kα, PI3Ky and mTOR. PP-121 is a multi-target inhibitor of PDGFR, Hck, mTOR, VEGFR2, Src and Abl. OSI-027 is a selective and potent dual inhibitor of mTORCI and mTORC2 with IC50 of 22 nM and 65 nM, respectively. Palomid 529 is a small molecule inhibitor of mTORC1 that lacks affinity for ABCB1/ABCG2 and has good brain penetration (Lin et al. (2013) Int J Cancer DOI: 10.1002/ijc. 28126 (e-published ahead of print). PP242 is a selective mTOR inhibitor. XL765 is a dual inhibitor of mTOR/PI3k for mTOR, p110α, p110β, p110γ and p110δ. GSK1059615 is a novel and dual inhibitor of PI3Kα, PI3 Kβ, PI3Kδ, PI3Kγ and mTOR. WYE-354 inhibits mTORC1 in HEK293 cells (0.2 μM-5 μM) and in HUVEC cells (10 nM-1 μM). WYE-354 is a potent, specific and ATP-competitive inhibitor of mTOR. Deforolimus (Ridaforolimus, AP23573, MK-8669) is a selective mTOR inhibitor.

### B. Other Components in the Nanoparticle Composition

In some embodiments, the composition is suitable for administration to a human. In some embodiments, the composition is suitable for administration to a mammal such as, in the veterinary context, domestic pets and agricultural animals. The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil. The formulations can additionally include lubricating agents, wetting agents, emulsifying and suspending agents, preserving agents, sweetening agents or flavoring agents.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. Injectable formulations are preferred.

In some embodiments, the composition is formulated to have a pH range of about 4.5 to about 9.0, including for example pH ranges of about any of 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 8). The composition can also be made to be isotonic with blood by the addition of a suitable tonicity modifier, such as glycerol.

### C. Albumin-Based Nanoparticle Compositions of Rapamycin

The methods described herein are particularly suitable for albumin-based nanoparticle compositions described herein in more details. The nanoparticle composition in some embodiments includes (a) nanoparticles that include rapamycin and albumin, and (b) a non-nanoparticle portion that includes rapamycin and albumin. The rapamycin and the albumin of the nanoparticles are associated with each other in the nanoparticles. For example, the nanoparticles may include a coating having the albumin, which surrounds a core comprising the rapamycin. In the non-nanoparticle portion of the composition, the rapamycin and the albumin may or may not associated with each other (i.e., the rapamycin may be in a reversible binding equilibrium with the albumin), but do not associate with each other in a manner that forms nanoparticles. That is, the nanoparticle composition may include nanoparticle-bound albumin and nanoparticle-bound rapamycin in the nanoparticle portion of the composition, and non-nanoparticle albumin and non-nanoparticle rapamycin in the non-nanoparticle portion of the composition. As used herein, “in the nanoparticles” is used synonymously with “in the nanoparticle portion.” The albumin of the nanoparticles may be further distinguishable from the albumin in the non-nanoparticle portion of the composition; for example, the oligomeric profile of the albumin in the nanoparticles may differ from the oligomeric profile of the albumin in the non-nanoparticle portion of the composition. The oligomer profile means the percentage of various albumin species compared with the total albumin in the composition. The types of albumin species includes albumin monomers, dimers, trimers, oligomers, and polymers. As used herein, “albumin monomers” or “monomeric albumin” refers to an albumin species having one, and only one, albumin unit; “albumin dimers” or “dimeric albumin” refers to an albumin species having two, and only two, albumin units; “albumin trimers” or “trimeric albumin” refers to albumin species having three, and only three, albumin units; “albumin polymers” refers to albumin species having a higher molecular weight than albumin monomers and albumin dimers; “albumin oligomers” or “oligomeric albumin” refers to lower molecular weight polymeric albumin species associated with a UV-based size-exclusion chromatography peak observed between a peak associated with albumin dimers and higher molecular weight polymeric albumin species.

The albumin of the nanoparticles associates with the rapamycin of the nanoparticles so that a nanoparticle suspension has a high concentration of rapamycin, which allows the composition to be used as a pharmaceutical composition for treating certain diseases, such as cancer. Manufactured nanoparticles (which may be made, for example, using the methods described herein) may be formulated, filtered, or otherwise processed to obtain the pharmaceutical composition, which may be suitable for medical use in a human individual.

Generally, to make the rapamycin pharmaceutical compositions described herein, rapamycin is dissolved in an organic solvent. Suitable organic solvents include, for example, ketones, esters, ethers, chlorinated solvents, and other solvents known in the art. For example, the organic solvent can be a mixture of methylene chloride/ethanol, chloroform/ethanol, or chloroform/tert-butanol (for example with a ratio of about any one of 1:9, 1:8, 1:7, 1:6, 1:5, 1:4, 1:3, 1:2, 1:1, 2:1, 3:1, 4:1, 5:1, 6:1, 7:1, 8:1, or 9:1 or with a ratio of about any one of 3:7, 5:7, 4:6, 5:5, 6:5, 8:5, 9:5, 9.5:5, 5:3, 7:3, 6:4, or 9.5:0.5). In some embodiments, the organic solvent comprises between about 10% and about 50% tert-butanol by volume. In some embodiments, the organic solvent comprises about any of 10%, 15%, 20%, 25%, 30%, 35%, 40%, 45%, or 50% tert-butanol by volume. In some embodiments, the organic solvent comprises about any of 10-15%, 15-20%, 20-25%, 25-30%, 30-35%, 35-40%, 40-45%, or 45-50%, or any combination of such ranges, of tert-butanol by volume. In some embodiments, the organic solvent comprises between about 50% and about 90% chloroform by volume. In some embodiments, the organic solvent comprises about any of 50%, 55%, 60%, 65%, 70%, 75%, 80%, 85%, or 90% chloroform by volume. In some embodiments, the organic solvent comprises about any of 50-55%, 55-60%, 60-65%, 65-70%, 70-75%, 75-80%, 80-85%, or 85-90%, or any combination of such ranges, of chloroform by volume. In some embodiments, the organic solvent comprises between about 10% and about 50% tert-butanol by volume and between about 50% and about 90% chloroform by volume. In some embodiments, the organic solvent comprises chloroform and tert-butanol at a volumetric ratio of about 1:1 to about 1:9, such as about any of 1:1, 2:1, 3:1, 4:1, 5:1, 6:1, 7:1, 8:1, and 9:1.

Albumin (such as recombinant albumin, for example NOVOZYME™ recombinant albumin or INTRIVIA™ recombinant albumin disclosed herein) is dissolved in an aqueous solution (such as water) and combined with the rapamycin solution to form a crude emulsion. The mixture is subjected to high pressure homogenization (e.g., using an Avestin, APV Gaulin, MICROFLUIDIZER™ such as a MICROFLUIDIZER™ Processor M-110EH from Microfluidics, Stansted, or Ultra Turrax homogenizer). The emulsion may be cycled through the high pressure homogenizer for between about 2 to about 100 cycles, such as about 5 to about 50 cycles or about 6 to about 20 cycles (e.g., about any one of 6, 8, 10, 12, 14, 16, 18 or 20 cycles). The organic solvent can then be removed by evaporation utilizing suitable equipment known for this purpose, including, but not limited to, rotary evaporators, falling film evaporators, wiped film evaporators, spray driers, and the like that can be operated in batch mode or in continuous operation. In some embodiments, the evaporator is a wiped film evaporator. The solvent may be removed at reduced pressure (such as at about any one of 25 mm Hg, 30 mm Hg, 40 mm Hg, 50 mm Hg, 100 mm Hg, 200 mm Hg, or 300 mm Hg). The amount of time used to remove the solvent under reduced pressure may be adjusted based on the volume of the formulation. For example, for a formulation produced on a 300 mL scale, the solvent can be removed at about 1 to about 300 mm Hg (e.g., about any one of 5-100 mm Hg, 10-50 mm Hg, 20-40 mm Hg, or 25 mm Hg) for about 5 to about 60 minutes (e.g., about any one of 7, 8, 9, 10, 11, 12, 13, 14, 15 16, 18, 20, 25, or 30 minutes). The dispersion obtained can be further lyophilized.

The nanoparticle compositions described herein (such a pharmaceutical composition) may have distinct characteristics for any one or more (in any combination) of the following: (1) the oligomeric status of the albumin associated with (such as in) the nanoparticles, such as the percentage of albumin monomers, dimers, and/or polymers (or trimers) of the albumin associated with (such as in) the nanoparticles; (2) the oligomeric status of the albumin associated with (such as in) the non-nanoparticle portion of the composition, such as the percentage of albumin monomers, dimers, and/or polymers (or trimers) of the albumin associated with (such as in) the non-nanoparticle portion of the composition; (3) the oligomeric status of the total albumin in the composition, such as the percentage of albumin monomers, dimers, and/or polymers (or trimers) of the total albumin in the composition; (4) the particle size profile of the nanoparticles, such as the average particle size, polydispersity index, and/or size distribution; (5) the portion (e.g., weight percentage) of the nanoparticles that is albumin and/or the portion (e.g., weight percentage) of the nanoparticles that is rapamycin; (6) the weight ratio of the albumin to the rapamycin in the nanoparticles; (7) the weight ratio of the albumin to the rapamycin in the non-nanoparticle portion of the composition; (8) the weight ratio of the albumin to the rapamycin in the non-nanoparticle portion of the composition (9) the weight ratio of the total albumin to the total rapamycin in the composition; (10) the portion (e.g., weight percentage) of rapamycin that is in the nanoparticles (or the non-nanoparticle portion of the composition) compared to the total rapamycin in the composition; (11) the portion (e.g., weight percentage) of albumin that is in the non-nanoparticle portion (or in the nanoparticles) compared to the total albumin in the composition; (12) the concentration of albumin in the composition; (13) the concentration of albumin in the non-nanoparticle portion of the composition; (14) the concentration of albumin in the composition that is associated with (such as in) the nanoparticles; (15) the concentration of rapamycin in the composition; (16) the concentration of rapamycin in the non-nanoparticle portion of the composition; (17) the concentration of rapamycin in the composition that is associated with (such as in) the nanoparticles; (18) the osmolality of the composition; (19) the viscosity of the composition; (20) the pH of the composition; (21) the stability of the nanoparticles in the composition; (22) the amount of residual solvent in the composition; (23) the zeta potential of the nanoparticles in the composition; (24) the crystalline status of the rapamycin in the nanoparticles; (25) the particle morphology of the nanoparticles, such as the shape, sphericity, thickness of the coating, and/or surface-to-volume ratio; (26) the weight percentage of seco-rapamycin in the nanoparticles, as compared to the sum of seco-rapamycin and rapamycin, by weight; (27) the presence, percentage, or concentration of albumin stabilizer (such as sodium caprylate and/or N-acetyltryptophanate) in the composition; (28) the recovery of rapamycin following filtration; (29) in vitro release kinetics of the nanoparticles; (30) the portion of total rapamycin in the composition that is both in the non-nanoparticle portion of the composition and not bound to albumin; and/or (31) the weight percentage of seco-rapamycin in the composition, as compared to the sum of seco-rapamycin and rapamycin, by weight. In some embodiments, the oligomeric status (such as the percentage of albumin monomers, dimers, or polymers (or trimers)) of the nanoparticles, the non-nanoparticles portion, or the total composition is assessed by size-exclusion chromatography using a saline mobile phase coupled with a multiple angle light scattering (MALS) detector).

The nanoparticle compositions described herein (such a pharmaceutical composition) may have distinct characteristics for any one or more (in any combination) of the following: (1) the oligomeric status of the albumin associated with (such as in) the nanoparticles, such as the percentage of albumin monomers, dimers, oligomers, and/or polymers (other than oligomers) of the albumin associated with (such as in) the nanoparticles; (2) the oligomeric status of the albumin associated with (such as in) the non-nanoparticle portion of the composition, such as the percentage of albumin monomers, dimers, oligomers, and/or polymers (other than oligomers) of the albumin associated with (such as in) the non-nanoparticle portion of the composition; (3) the oligomeric status of the total albumin in the composition, such as the percentage of albumin monomers, dimers, oligomers, and/or polymers (other than oligomers) of the total albumin in the composition; (4) the particle size profile of the nanoparticles, such as the average particle size, polydispersity index, and/or size distribution; (5) the portion (e.g., weight percentage) of the nanoparticles that is albumin and/or the portion (e.g., weight percentage) of the nanoparticles that is rapamycin; (6) the weight ratio of the albumin to the rapamycin in the nanoparticles; (7) the weight ratio of the albumin to the rapamycin in the non-nanoparticle portion of the composition; (8) the weight ratio of the albumin to the rapamycin in the non-nanoparticle portion of the composition (9) the weight ratio of the total albumin to the total rapamycin in the composition; (10) the portion (e.g., weight percentage) of rapamycin that is in the nanoparticles (or the non-nanoparticle portion of the composition) compared to the total rapamycin in the composition; (11) the portion (e.g., weight percentage) of albumin that is in the non-nanoparticle portion (or in the nanoparticles) compared to the total albumin in the composition; (12) the concentration of albumin in the composition; (13) the concentration of albumin in the non-nanoparticle portion of the composition; (14) the concentration of albumin in the composition that is associated with (such as in) the nanoparticles; (15) the concentration of rapamycin in the composition; (16) the concentration of rapamycin in the non-nanoparticle portion of the composition; (17) the concentration of rapamycin in the composition that is associated with (such as in) the nanoparticles; (18) the osmolality of the composition; (19) the viscosity of the composition; (20) the pH of the composition; (21) the stability of the nanoparticles in the composition; (22) the amount of residual solvent in the composition; (23) the zeta potential of the nanoparticles in the composition; (24) the crystalline status of the rapamycin in the nanoparticles; (25) the particle morphology of the nanoparticles, such as the shape, sphericity, thickness of the coating, and/or surface-to-volume ratio; (26) the weight percentage of seco-rapamycin in the nanoparticles, as compared to the sum of seco-rapamycin and rapamycin, by weight; (27) the presence, percentage, or concentration of albumin stabilizer (such as sodium caprylate and/or N-acetyltryptophanate) in the composition; (28) the recovery of rapamycin following filtration; (29) in vitro release kinetics of the nanoparticles; (30) the portion of total rapamycin in the composition that is both in the non-nanoparticle portion of the composition and not bound to albumin; and/or (31) the weight percentage of seco-rapamycin in the composition, as compared to the sum of seco-rapamycin and rapamycin, by weight. As used herein, “albumin oligomers” or “oligomeric albumin” refers to lower molecular weight polymeric albumin species associated with a UV-absorbance-based size-exclusion chromatography peak observed between a peak associated with albumin dimers and higher molecular weight polymeric albumin species. In some embodiments, the oligomeric status (such as the percentage of albumin monomers, dimers, oligomers, or polymers (other than oligomers)) of the nanoparticles, the non-nanoparticle portion, or the total composition is assessed by size-exclusion chromatography using a mobile phase containing an aqueous portion and a miscible organic portion (such as an aqueous buffer containing 7.5% methanol) coupled with a UV detector. In some embodiments, the percentage of albumin in the nanoparticle portion that is in the form of monomeric, dimeric, oligomeric, or polymeric albumin (other than oligomeric albumin) is determined by separating the nanoparticles from the non-nanoparticle portion, dissolving the nanoparticles, and subjecting the dissolved nanoparticles to size-exclusion chromatography. In some embodiments, the size-exclusion chromatography uses a mobile phase containing an aqueous portion and a miscible organic portion (such as an aqueous buffer containing 7.5% methanol) coupled with a UV detector.

In some embodiments, the nanoparticle composition has one or more of the following distinct characteristics: (1) about 80% to about 95% (or as further provided herein) of the total albumin in the composition is in the form of monomeric albumin; (2) about 4% to about 15% (or as further provided herein) of the total albumin in the composition is in the form of dimeric albumin; (3) about 0.5% to about 5% (or as further provided herein) of the total albumin in the composition is in the form of polymeric albumin (or trimeric albumin); (4) the weight ratio of the total albumin to the total rapamycin in the composition is about 1:1 to about 10:1 (or as further provided herein); (5) about 90% or more (or as further provided herein) of the total rapamycin in the composition is in the nanoparticles; (6) about 90% or more (or as further provided herein) of the total albumin in the composition is in the non-nanoparticle portion of the nanoparticles; (7) the composition comprises tert-butanol at a concentration of less than about 10 μg/mL or less than about 10 ppm (or as further provided herein); (8) the composition comprises chloroform at a concentration of less than about 5 μg/mL or less than about 5 ppm (or as further provided herein); (9) the composition comprises an albumin stabilizer (such as sodium caprylate and/or N-acetyltryptophanate); (10) at least about 80% or more (or as further provided herein) of the rapamycin in the composition is recoverable after filtering the composition with a 0.2 micron filter; (11) the composition is stable for at least 24 hours; and/or (12) less than about 5% of the total rapamycin in the composition is both in the non-nanoparticle portion of the composition and unbound to albumin in the non-nanoparticle portion of the composition. In some embodiments, the nanoparticle composition may be a nanoparticle suspension, and the nanoparticle composition may have one or more of the following distinct characteristics (in addition to or in alternative to any one of the previously described district characteristics): (1) the concentration of albumin in the composition is about 30 mg/mL to about 100 mg/mL (or as further provided herein); (2) the concentration of rapamycin in the composition is about 1 mg/mL to about 15 mg/mL (or as further provided herein, such as about 1 mg/mL to about 7 mg/mL); (3) the osmolality of the composition is about 300 mOsm/kg to about 350 mOsm/kg (or as otherwise provided herein); (4) the viscosity of the composition is about 1.2 cP to about 1.5 cP (or as otherwise provided herein); and/or (5) the pH of the composition is about 6.0 to about 7.5 (or as otherwise provided herein).

In some embodiments, the nanoparticles of the composition have one or more of the following distinct characteristics: (1) about 70% to about 85% (or as otherwise provided herein) of the albumin in the nanoparticles is in the form of albumin monomers; (2) about 9% to about 20% (or as otherwise provided herein) of the albumin in the nanoparticles is in the form of albumin dimers; (3) about 5% to about 15% (or as otherwise provided herein) of the albumin in the nanoparticles is in the form of albumin polymers (or albumin trimers); (4) the nanoparticles have a volume weighted mean particle size and/or Z-average particle size of about 200 nm or less (or as otherwise provided herein, such as between about 50 nm and about 200 nm); (5) the nanoparticles have a polydispersity index of less than about 0.2 (or as otherwise provided herein, such as between about 0.03 and about 0.2); (6) the span of the particle size distribution ((Dv95-Dv5)/Dv50) is about 0.8 to about 1.2 (or as otherwise provided herein); (7) the nanoparticles are about 25% to about 45% albumin by weight (or as otherwise provided herein); (8) the nanoparticles are about 55% to about 75% rapamycin by weight (or as otherwise provided herein); (9) the weight ratio of albumin to rapamycin in the nanoparticles is about 1:1 to about 1:4 (or as otherwise provided herein); (10) the zeta potential of the nanoparticles in the composition is about −25 mV to about −50 mV (or as otherwise provided herein); (11) the nanoparticles have an amorphous morphology; (12) the rapamycin in the nanoparticles has an amorphous morphology; (13) the vinyl chain of the rapamycin in the nanoparticles interacts with the albumin in the nanoparticles; (14) at least a portion (such as at least 20%, or as otherwise provided herein) of the nanoparticles in the composition are non-spherical; (15) the nanoparticles comprise less than about 2.5% seco-rapamycin (or as otherwise provided herein, such as between about 0.2% and about 2.5%) compared to the sum of seco-rapamycin and rapamycin by weight; and/or (16) the composition comprises less than 3% seco-rapamycin (or as otherwise provided herein, such as between about 0.2% and about 2.5%) compared to the sum of seco-rapamycin and rapamycin by weight. In some embodiments, the nanoparticle composition may be a nanoparticle suspension, and in some embodiments the concentration of the albumin in the nanoparticle suspension that is in the nanoparticles is about 1.8 mg/mL to about 3 mg/mL (or as otherwise provided herein).

In some embodiments, the nanoparticles of the composition have one or more of the following distinct characteristics: (1) about 25% to about 50% (or as otherwise provided herein) of the albumin in the nanoparticles is in the form of albumin monomers; (2) about 5% to about 16% (or as otherwise provided herein) of the albumin in the nanoparticles is in the form of albumin dimers; (3) about 1% to about 4.5% (or as otherwise provided herein) of the albumin in the nanoparticles is in the form of albumin oligomers; (4) about 42% to about 60% (or as otherwise provided herein) of the albumin in the nanoparticles is in the form of albumin polymers (other than oligomers); (5) the nanoparticles have a volume weighted mean particle size and/or Z-average particle size of about 200 nm or less (or as otherwise provided herein, such as between about 50 nm and about 200 nm); (6) the nanoparticles have a polydispersity index of less than about 0.2 (or as otherwise provided herein, such as between about 0.03 and about 0.2); (7) the span of the particle size distribution ((Dv95-Dv5)/Dv50) is about 0.8 to about 1.2 (or as otherwise provided herein); (8) the nanoparticles are about 25% to about 45% albumin by weight (or as otherwise provided herein); (9) the nanoparticles are about 55% to about 75% rapamycin by weight (or as otherwise provided herein); (10) the weight ratio of albumin to rapamycin in the nanoparticles is about 1:1 to about 1:4 (or as otherwise provided herein); (11) the zeta potential of the nanoparticles in the composition is about −25 mV to about −50 mV (or as otherwise provided herein); (12) the nanoparticles have an amorphous morphology; (13) the rapamycin in the nanoparticles has an amorphous morphology; (14) the vinyl chain of the rapamycin in the nanoparticles interacts with the albumin in the nanoparticles; (15) at least a portion (such as at least 20%, or as otherwise provided herein) of the nanoparticles in the composition are non-spherical; (16) the nanoparticles comprise less than about 2.5% seco-rapamycin (or as otherwise provided herein, such as between about 0.2% and about 2.5%) compared to the sum of seco-rapamycin and rapamycin by weight; and/or (17) the composition comprises less than about 3% seco-rapamycin (or as otherwise provided herein, such as between about 0.2% and about 3%) compared to the sum of seco-rapamycin and rapamycin, by weight. In some embodiments, the nanoparticle composition may be a nanoparticle suspension, and in some embodiments the concentration of the albumin in the nanoparticle suspension that is in the nanoparticles is about 1.8 mg/mL to about 3 mg/mL (or as otherwise provided herein).

In some embodiments, the non-nanoparticle portion of the composition has one or more of the following distinct characteristics: (1) about 80% to about 95% (or as otherwise provided herein) of the albumin in the non-nanoparticle portion of the composition is in the form of albumin monomers; (2) about 5% to about 14% (or as otherwise provided herein) of the albumin in the non-nanoparticle portion of the composition is in the form of albumin dimers; and/or (3) about 1% to about 5% (or as otherwise provided herein) of the albumin in the non-nanoparticle portion of the composition is in the form of albumin polymers (or albumin trimers). In some embodiments, the nanoparticle composition may be a nanoparticle suspension, and the non-nanoparticle portion of the nanoparticle suspension may have one or more of the following distinct characteristics (in addition to or in alternative to any one of the previously described district characteristics): (1) the concentration of albumin in the non-nanoparticle portion of the composition is between about 30 mg/mL and about 100 mg/mL (or as otherwise provided herein); and/or (2) the concentration of rapamycin in the non-nanoparticle portion is about 20 μg/mL to about 55 μg/mL (or as otherwise provided herein).

In some embodiments, the non-nanoparticle portion of the composition has one or more of the following distinct characteristics: (1) about 80% to about 95% (or as otherwise provided herein) of the albumin in the non-nanoparticle portion of the composition is in the form of albumin monomers; (2) about 5% to about 16% (or as otherwise provided herein) of the albumin in the non-nanoparticle portion of the composition is in the form of albumin dimers; about 0.5% to about 4% (or as otherwise provided herein) of the albumin in the non-nanoparticle portion of the composition is in the form of albumin oligomers; and/or (4) about 0.5% to about 3% (or as otherwise provided herein) of the albumin in the non-nanoparticle portion of the composition is in the form of albumin polymers (other than oligomers). In some embodiments, the nanoparticle composition may be a nanoparticle suspension, and the non-nanoparticle portion of the nanoparticle suspension may have one or more of the following distinct characteristics (in addition to or in alternative to any one of the previously described district characteristics): (1) the concentration of albumin in the non-nanoparticle portion of the composition is between about 30 mg/mL and about 100 mg/mL (or as otherwise provided herein); and/or (2) the concentration of rapamycin in the non-nanoparticle portion is about 20 μg/mL to about 55 μg/mL (or as otherwise provided herein).

The compositions (such as pharmaceutical compositions) described herein can be in liquid (e.g., as a nanoparticle suspension) or powder forms. For example, in some embodiments, the composition is a liquid nanoparticle suspension (for example prior to lyophilization). In some embodiments, the composition is a reconstituted suspension (e.g., in an aqueous solution such as a saline solution). In some embodiments, the composition is dried, such as lyophilized. In some embodiments, the composition is sterile. In some embodiments, the composition is contained in a sealed container, such as a sealed vial (e.g., a glass vial) or sealed bag.

In some embodiments, the nanoparticle composition comprises (a) nanoparticles comprising rapamycin and albumin (such as human albumin), and (b) a non-nanoparticle portion comprising albumin (such as human albumin) and rapamycin. In some embodiments, about 0.5% to about 5% of the albumin in the non-nanoparticle portion or the total albumin in the nanoparticle composition is in the form of polymeric albumin (or trimeric albumin). In some embodiments, about 4% to about 14% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles comprising rapamycin and albumin (such as human albumin), wherein about 70% to about 85% of the albumin in the nanoparticles is in the form of monomeric albumin; and (b) a non-nanoparticle portion comprising albumin (such as human albumin) and rapamycin.

In some embodiments, the nanoparticle composition comprises (a) nanoparticles comprising rapamycin and albumin (such as human albumin), wherein about 25% to about 50% of the albumin in the nanoparticles is in the form of monomeric albumin; and (b) a non-nanoparticle portion comprising albumin (such as human albumin) and rapamycin.

In some embodiments, the nanoparticle composition comprises (a) nanoparticles comprising rapamycin and albumin (such as human albumin), wherein about 5% to about 15% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles comprising rapamycin and albumin (such as human albumin), wherein about 25% to about 50% of the albumin in the nanoparticles is in the form of polymeric albumin (other than oligomeric albumin); and (b) a non-nanoparticle portion comprising albumin (such as human albumin) and rapamycin.

In some embodiments, the nanoparticle composition comprises (a) nanoparticles comprising rapamycin and albumin (such as human albumin), wherein about 9% to about 20% of the albumin in the nanoparticles is in the form of dimeric albumin; and (b) a non-nanoparticle portion comprising albumin (such as human albumin) and rapamycin.

In some embodiments, the nanoparticle composition comprises (a) nanoparticles comprising rapamycin and albumin (such as human albumin), wherein about 5% to about 16% of the albumin in the nanoparticles is in the form of dimeric albumin; and (b) a non-nanoparticle portion comprising albumin (such as human albumin) and rapamycin.

In some embodiments, the nanoparticle composition comprises (a) nanoparticles comprising rapamycin and albumin (such as human albumin), wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles comprising rapamycin and albumin (such as human albumin), wherein about 25% to about 50% of the albumin in the nanoparticles is in the form of monomeric albumin, about 1% to about 4.5% of the albumin in the nanoparticles is in the form of oligomeric albumin, about 5% to about 16% of the albumin in the nanoparticles is in the form of dimeric albumin, and about 25% to about 50% of the albumin in the nanoparticles is in the form of polymeric albumin (other than oligomeric albumin); and (b) a non-nanoparticle portion comprising albumin (such as human albumin) and rapamycin.

In some embodiments, the nanoparticle composition comprises (a) nanoparticles having a Z-average particle size of about 200 nm or less (such as about 50 nm to about 200 nm), comprising rapamycin and albumin (such as human albumin), wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles having a Z-average particle size of about 200 nm or less (such as about 50 nm to about 200 nm), comprising a coating comprising albumin (such as human albumin) and a core comprising rapamycin, wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles having a Z-average particle size of about 200 nm or less (such as about 50 nm to about 200 nm), comprising about 55% to about 65% (by weight) rapamycin and about 25% to about 45% (by weight) albumin (such as human albumin), wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles having a Z-average particle size of about 200 nm or less (such as about 50 nm to about 200 nm), comprising a coating comprising albumin (such as human albumin) and a core comprising rapamycin, wherein the albumin comprises about 25% to about 45% of the nanoparticles by weight and the rapamycin comprises about 55% to about 75% of the nanoparticles by weight, wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles having a Z-average particle size of about 200 nm or less (such as about 50 nm to about 200 nm), comprising about 55% to about 75% (by weight) rapamycin and about 25% to about 45% (by weight) albumin (such as human albumin), wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL).

In some embodiments, the nanoparticle composition comprises (a) nanoparticles having a Z-average particle size of about 200 nm or less (such as about 50 nm to about 200 nm), comprising a coating comprising albumin (such as human albumin) and a core comprising rapamycin, wherein the albumin comprises about 25% to about 45% of the nanoparticles by weight and the rapamycin comprises about 55% to about 75% of the nanoparticles by weight, wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL).

In some embodiments, the nanoparticle composition comprises (a) nanoparticles having a Z-average particle size of about 200 nm or less (such as about 50 nm to about 200 nm) and a zeta potential of about −25 mV to about −50 mV, comprising about 55% to about 75% (by weight) rapamycin and about 25% to about 45% (by weight) albumin (such as human albumin), wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL).

In some embodiments, the nanoparticle composition comprises (a) nanoparticles having a Z-average particle size of about 200 nm or less (such as about 50 nm to about 200 nm) and a zeta potential of about −25 mV to about −50 mV, comprising a coating comprising albumin (such as human albumin) and a core comprising rapamycin, wherein the albumin comprises about 25% to about 45% of the nanoparticles by weight and the rapamycin comprises about 55% to about 75% of the nanoparticles by weight, wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL).

In some embodiments, the nanoparticle composition comprises (a) nanoparticles having a Z-average particle size of about 200 nm or less (such as about 50 nm to about 200 nm) and a zeta potential of about −25 mV to about −50 mV, comprising about 55% to about 75% (by weight) rapamycin and about 25% to about 45% (by weight) albumin (such as human albumin), wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL); and wherein about 3% or less of the rapamycin in the nanoparticle composition is free rapamycin.

In some embodiments, the nanoparticle composition comprises (a) nanoparticles having a Z-average particle size of about 200 nm or less (such as about 50 nm to about 200 nm) and a zeta potential of about −25 mV to about −50 mV, comprising a coating comprising albumin (such as human albumin) and a core comprising rapamycin, wherein the albumin comprises about 25% to about 45% of the nanoparticles by weight and the rapamycin comprises about 55% to about 75% of the nanoparticles by weight, wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL); and wherein about 3% or less of the rapamycin in the nanoparticle composition is free rapamycin.

In some embodiments, the nanoparticle composition comprises (a) nanoparticles having a Z-average particle size of about 200 nm or less (such as about 50 nm to about 200 nm) and a zeta potential of about −25 mV to about −50 mV, comprising about 55% to about 75% (by weight) rapamycin and about 25% to about 45% (by weight) albumin (such as human albumin), wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL); and wherein the sum of seco-rapamycin and rapamycin in the nanoparticles is less than 3% (such as about 0.2% to about 3%) seco-rapamycin, by weight. In some embodiments, the sum of seco-rapamycin and rapamycin in the composition is less than 3% (such as about 0.2% to about 3%) seco-rapamycin, by weight.

In some embodiments, the nanoparticle composition comprises (a) nanoparticles having a Z-average particle size of about 200 nm or less (such as about 50 nm to about 200 nm) and a zeta potential of about −25 mV to about −50 mV, comprising a coating comprising albumin (such as human albumin) and a core comprising rapamycin, wherein the albumin comprises about 25% to about 45% of the nanoparticles by weight and the rapamycin comprises about 55% to about 75% of the nanoparticles by weight, wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL); and wherein the sum of seco-rapamycin and rapamycin in the nanoparticles is less than 3% (such as about 0.2% to about 3%) seco-rapamycin, by weight. In some embodiments, the seco-rapamycin is less than 3% (such as about 0.2% to about 3%) of the sum of seco-rapamycin and rapamycin in the composition.

In some embodiments, the nanoparticle composition comprises (a) nanoparticles comprising rapamycin and albumin (such as human albumin), wherein about 74% to about 80% of the albumin in the nanoparticles is in the form of monomeric albumin; and (b) a non-nanoparticle portion comprising albumin (such as human albumin) and rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL.

In some embodiments, the nanoparticle composition comprises (a) nanoparticles comprising rapamycin and albumin (such as human albumin), wherein about 7% to about 11% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles comprising rapamycin and albumin (such as human albumin), wherein about 12% to about 17% of the albumin in the nanoparticles is in the form of dimeric albumin; and (b) a non-nanoparticle portion comprising albumin (such as human albumin) and rapamycin.

In some embodiments, the nanoparticle composition comprises (a) nanoparticles comprising rapamycin and albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles having a Z-average particle size of about 85 nm to about 95 nm, comprising rapamycin and albumin (such as human albumin); and (b) a non-nanoparticle portion comprising albumin (such as human albumin) and rapamycin.

In some embodiments, the nanoparticle composition comprises (a) nanoparticles having a Z-average particle size of about 85 nm to about 95 nm, comprising rapamycin and albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles having a Z-average particle size of about 85 nm to about 95 nm, comprising a coating comprising albumin (such as human albumin) and a core comprising rapamycin, wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles having a zeta potential of about −33 mV to about −39 mV, comprising rapamycin and albumin (such as human albumin); and (b) a non-nanoparticle portion comprising albumin (such as human albumin) and rapamycin.

In some embodiments, the nanoparticle composition comprises (a) nanoparticles having a zeta potential of about −33 mV to about −39 mV, comprising a coating comprising albumin (such as human albumin) and a core comprising rapamycin; and (b) a non-nanoparticle portion comprising albumin (such as human albumin) and rapamycin

In some embodiments, the nanoparticle composition comprises (a) nanoparticles having a zeta potential of about −33 mV to about −39 mV, comprising rapamycin and albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles having a zeta potential of about −33 mV to about −39 mV, comprising a coating comprising albumin (such as human albumin) and a core comprising rapamycin, wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles having a Z-average particle size of about 85 nm to about 95 nm and a zeta potential of about −33 mV to about −39 mV, comprising rapamycin and albumin (such as human albumin); and (b) a non-nanoparticle portion comprising albumin (such as human albumin) and rapamycin.

In some embodiments, the nanoparticle composition comprises (a) nanoparticles having a Z-average particle size of about 85 nm to about 95 nm and a zeta potential of about −33 mV to about −39 mV, comprising a coating comprising albumin (such as human albumin) and a core comprising rapamycin; and (b) a non-nanoparticle portion comprising albumin (such as human albumin) and rapamycin

In some embodiments, the nanoparticle composition comprises (a) nanoparticles having a Z-average particle size of about 85 nm to about 95 nm and a zeta potential of about −33 mV to about −39 mV, comprising rapamycin and albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles having a Z-average particle size of about 85 nm to about 95 nm and a zeta potential of about −33 mV to about −39 mV, comprising a coating comprising albumin (such as human albumin) and a core comprising rapamycin, wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles having a Z-average particle size of about 85 nm to about 95 nm, comprising about 62% to about 68% (by weight) rapamycin and about 32% to about 38% (by weight) albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles having a Z-average particle size of about 85 nm to about 95 nm, comprising about 62% to about 68% (by weight) rapamycin and about 32% to about 38% (by weight) albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL).

In some embodiments, the nanoparticle composition comprises (a) nanoparticles having a Z-average particle size of about 85 nm to about 95 nm and a zeta potential of about −33 mV to about −39 mV, comprising about 62% to about 68% (by weight) rapamycin and about 32% to about 38% (by weight) albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL).

In some embodiments, the nanoparticle composition comprises (a) nanoparticles having a Z-average particle size of about 85 nm to about 95 nm and a zeta potential of about −33 mV to about −39 mV, comprising about 62% to about 68% (by weight) rapamycin and about 32% to about 38% (by weight) albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL); and wherein about 1% or less of the rapamycin in the nanoparticle composition is free rapamycin.

In some embodiments, the nanoparticle composition comprises (a) nanoparticles having a Z-average particle size of about 85 nm to about 95 nm and a zeta potential of about of about-33 mV to about −39 mV, comprising about 62% to about 68% (by weight) rapamycin and about 32% to about 38% (by weight) albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL); and wherein the sum of seco-rapamycin and rapamycin in the nanoparticles is less than 1% (such as about 0.5% to about 1%) seco-rapamycin, by weight. In some embodiments, seco-rapamycin is greater than about 0.2% (such as about 0.2% to about 3%) of the sum of seco-rapamycin and rapamycin in the composition.

Also provided herein are commercial batches of the nanoparticle compositions (such as the pharmaceutical compositions) for use of any one of the treatment methods described here. “Commercial batch” as used herein refers to a batch size that is at least about 20 grams (by mass of rapamycin). Commercial batches are produced at a larger scale than experimental or bench-scale batches. The increased scale is associated with longer production times, including longer steps (such as evaporation steps) or longer hold times between steps.

In some embodiments, the mTOR inhibitor nanoparticle composition (e.g., a sirolimus/albumin nanoparticle composition, such as FYARRO™) is administered subcutaneously. In some embodiments, the mTOR inhibitor nanoparticle composition (e.g., a sirolimus/albumin nanoparticle composition, such as FYARRO™) is administered intravenously. In some embodiments, the mTOR inhibitor nanoparticle composition (e.g., a sirolimus/albumin nanoparticle composition, such as FYARRO™) is administered at a dose between about 1 mg/m² and about 150 mg/m², between about 5 mg/m² and about 75 mg/m², e.g., via intravenous infusion. In some embodiments, the mTOR inhibitor nanoparticle composition (e.g., a sirolimus/albumin nanoparticle composition, such as FYARRO™) is administered at a dose of about any one of 5, 7.5, 10, 15, 30, 56, 75 or 100 mg/m², e.g., via intravenous infusion. In some embodiments, the mTOR inhibitor nanoparticle composition (e.g., a sirolimus/albumin nanoparticle composition, such as FYARRO™) is administered to the individual having cancer in one or more 21-day cycles (e.g., three-week cycles). In some embodiments, the mTOR inhibitor nanoparticle composition (e.g., a sirolimus/albumin nanoparticle composition, such as FYARRO™) is administered to the individual once during each 21-day cycle (e.g., three-week cycle). In some embodiments, the mTOR inhibitor nanoparticle composition (e.g., a sirolimus/albumin nanoparticle composition, such as FYARRO™) is administered during Week 1, Week 2, or Week 3 during each 21-day cycle (e.g., three-week cycle). In some embodiments, the mTOR inhibitor nanoparticle composition (e.g., a sirolimus/albumin nanoparticle composition, such as FYARRO™) is administered on Day 1, Day 8, or Day 15 of each 21-day cycle (e.g., three-week cycle). In some embodiments, the mTOR inhibitor nanoparticle composition (e.g., a sirolimus/albumin nanoparticle composition, such as FYARRO™) is administered to the individual twice during each 21-day cycle (e.g., three-week cycle). In some embodiments, the mTOR inhibitor nanoparticle composition (e.g., a sirolimus/albumin nanoparticle composition, such as FYARRO™) is administered during Week 1 and Week 2 during each 21-day cycle (e.g., three-week cycle). In some embodiments, the mTOR inhibitor nanoparticle composition (e.g., a sirolimus/albumin nanoparticle composition, such as FYARRO™) is administered during Week 2 and Week 3 during each 21-day cycle (e.g., three-week cycle). In some embodiments, the mTOR inhibitor nanoparticle composition (e.g., a sirolimus/albumin nanoparticle composition, such as FYARRO™) is administered during Week 1 and Week 3 during each 21-day cycle (e.g., three-week cycle). In some embodiments, the mTOR inhibitor nanoparticle composition (e.g., a sirolimus/albumin nanoparticle composition, such as FYARRO™) is administered on Day 1 and Day 8 of each 21-day cycle (e.g., three-week cycle). In some embodiments, the mTOR inhibitor nanoparticle composition (e.g., a sirolimus/albumin nanoparticle composition, such as FYARRO™) is administered on Day 1 and Day 15 of each 21-day cycle (e.g., three-week cycle). In some embodiments, the mTOR inhibitor nanoparticle composition (e.g., a sirolimus/albumin nanoparticle composition, such as FYARRO™) is administered on Day 8 and Day 15 of each 21-day cycle (e.g., three-week cycle). In some embodiments, the mTOR inhibitor nanoparticle composition (e.g., a sirolimus/albumin nanoparticle composition, such as FYARRO™) is administered to the individual three times during each 21-day cycle (e.g., three-week cycle). In some embodiments, the mTOR inhibitor nanoparticle composition (e.g., a sirolimus/albumin nanoparticle composition, such as FYARRO™) is administered during Week 1, Week 2, and Week 3 during each 21-day cycle (e.g., three-week cycle). In some embodiments, the mTOR inhibitor nanoparticle composition (e.g., a sirolimus/albumin nanoparticle composition, such as FYARRO™) is administered on Day 1, Day 8, and Day 15 of each 21-day cycle (e.g., three-week cycle). In some embodiments, the dosage of the mTOR inhibitor nanoparticle composition (e.g., a sirolimus/albumin nanoparticle composition, such as FYARRO™) is modified (e.g., if the individual experiences one or more adverse effects). Details regarding dosage modification for FYARRO™ and circumstances under which dosage modifications are made are detailed at www(dot)accessdata(dot)fda(dot)gov/drugsatfda_docs/label/2021/213312lbl.pdf.

### IV. Multi-Tyrosine Kinase Inhibitors

In some embodiments, the multi-tyrosine kinase inhibitor inhibits two or more (including any of 2, 3, 4, 5, 6, 7, 8, 9, 10, 11, or 12) of the following: vascular endothelial growth factor receptor (VEGFR)-1, -2, or -3, platelet endothelial growth factor receptor (PDGFR)-α or -β, interleukin-2 receptor-inducible T-cell kinase (ITK), leukocyte-specific protein tyrosine kinase (LCK), colony-stimulating factor-1 receptor (c-fms), fibroblast growth factor receptors (FGFR)-1, -3, or -4, or stem cell factor receptor c-Kit. In some embodiments, the multi-tyrosine kinase inhibitor inhibits vascular endothelial growth factor receptor (VEGFR)-1, -2, and -3, platelet endothelial growth factor receptor (PDGFR)-α and -β, interleukin-2 receptor-inducible T-cell kinase (ITK), leukocyte-specific protein tyrosine kinase (LCK), colony-stimulating factor-1 receptor (c-fms), fibroblast growth factor receptors (FGFR)-1, -3, and -4, and stem cell factor receptor c-Kit. Methods for determining inhibition, including inhibition of the features above, are understood in the art, and thus the scope of a multi-tyrosine kinase inhibitor described herein is also understood. For example, as described in Hamberg et al., Oncologist, 15, 2010; and Melichar et al., J Buon, 16, 2011, the contents of which are incorporated by reference herein in their entirety for all purposes.

In some embodiments, the multi-tyrosine kinase inhibitor competes for adenosine triphosphate for binding to the intracellular side of tyrosine kinase receptors and prevents the ATP-induced activation of said receptors. In some embodiments, the multi-tyrosine kinase inhibitor is an indazolylpyrimidine.

In some embodiments, the multi-tyrosine kinase inhibitor is pazopanib or a pharmaceutical salt thereof. In some embodiments, the multi-tyrosine kinase inhibitor is pazopanib hydrochloride (e.g., VOTRIENT®).

### V. Articles of Manufacture and Kits

In some embodiments, there is provided an article of manufacture containing materials useful for the treatment of a soft-tissue sarcoma (including spindle cell sarcoma, solitary fibrous tumor, or leiomyosarcoma), the article of manufacture, such as a medicament or medicament combination, comprising an mTOR inhibitor nanoparticle composition (such as sirolimus/albumin nanoparticle composition, e.g., nab-sirolimus) and a multi-tyrosine kinase inhibitor (e.g., pazopanib). The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example the container may be an intravenous solution bag or a vial having a stopper pierceable by a hypodermic injection needle). At least one active agent in the composition is (a) a nanoparticle formulation of an mTOR inhibitor (e.g., nab-sirolimus); or (b) a multi-tyrosine kinase inhibitor (e.g., pazopanib). The label or package insert indicates that the composition is used for treating the particular condition in an individual, such as described herein. The label or package insert will further comprise instructions for administering the composition to the individual according to the methods described herein. Articles of manufacture and kits comprising combination therapies described herein are also contemplated.

Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating a soft-tissue sarcoma (e.g., spindle cell sarcoma, solitary fibrous tumor, or leiomyosarcoma).

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for treatment of a soft-tissue sarcoma (e.g., spindle cell sarcoma, solitary fibrous tumor, or leiomyosarcoma). Kits of the invention include one or more containers comprising an mTOR inhibitor nanoparticle composition (such as sirolimus/albumin nanoparticle composition) (or unit dosage form and/or article of manufacture), and in some embodiments, further comprise a multi-tyrosine kinase inhibitor (e.g., pazopanib) and/or instructions for use in accordance with any of the methods described herein. The kit may further comprise a description of selection of individuals suitable for treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of the invention are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the mTOR inhibitor nanoparticle composition (such as sirolimus/albumin nanoparticle composition) and a multi-tyrosine kinase inhibitor (e.g., pazopanib) generally include information as to dosage, dosing schedule, and route of administration for the intended treatment. The containers may be unit doses, bulk packages (e.g., multi-dose packages) or sub-unit doses. For example, kits may be provided that contain sufficient dosages of an mTOR inhibitor nanoparticle composition (such as sirolimus/albumin nanoparticle composition) and a multi-tyrosine kinase inhibitor (e.g., pazopanib) as disclosed herein to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the mTOR inhibitor nanoparticle composition (such as sirolimus/albumin nanoparticle composition) and a multi-tyrosine kinase inhibitor (e.g., pazopanib) and instructions for use, packaged in quantities sufficient for storage and use in pharmacies, for example, hospital pharmacies and compounding pharmacies.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXAMPLES

### Example 1

This example demonstrates a phase I study administering nab-sirolimus and pazopanib to patients having a soft-tissue sarcoma (STS).

### Patients and Methods

### Study Design

This was a phase I study designed to determine the safety and feasibility of a combination of oral pazopanib hydrochloride and intravenous nab-sirolimus. To obtain preliminary information regarding efficacy, the study had limited enrollment to those patients with advanced/unresectable STS for whom single agent pazopanib was indicated as a therapy.

The primary endpoint of the study was the determination of a maximum-tolerated dose (MTD) and recommended Phase 2 dose of nab-sirolimus combined with pazopanib. Secondary endpoints included characterization of adverse events of the nab-sirolimus/pazopanib combination and descriptive characterization of clinical benefit of the nab-sirolimus/pazopanib combination (objective responses, progression-free survival duration, and clinical benefit rates at 3 and 6 months). Correlative endpoints were included to evaluate the correlation of clinical benefit with baseline mTOR pathway activation status, as assessed by immunohistochemistry for phosphorylated-S6 ribosomal protein, correlation of clinical benefit with baseline tumoral expression of Serum Protein Acidic and Rich in Cysteine (SPARC)/Osteonectin, as assessed by immunohistochemistry, and pharmacokinetics of nab-sirolimus in combination with pazopanib.

The study employed the standard “cohort of three” design (also commonly known as 3+3), wherein three patients were treated at each dose level with expansion to six patients per cohort when dose-limiting toxicity (DLT) was observed in one out of the three initially enrolled patients at each dose level. If no DLT occurred after two doses, escalation to the next dose level was permitted. The MTD was defined as the highest safely tolerated dose at which no more than one patient experienced DLT, with the next higher dose level having at least two patients who experienced DLT. Patients in the dose-escalation study were able to continue treatment at their designated dose levels until significant disease progression or unacceptable toxicity occurred. No intra-patient dose escalation took place. Dosage limiting toxicity was defined as Grade 3-5 adverse events according to Common Terminology Criteria for Adverse Events version 5.0 occurring during cycle 1 of treatment.

Pazopanib was administered by mouth and nab-sirolimus was administered intravenously. Initial cohorts received 800 mg or 400 mg pazopanib daily and 60, 45, or 30 mg/m² of nab-sirolimus on days 1 and 8 of a 21-day cycle. Subsequently, nab-sirolimus was administered only on day 1 of a 21-day cycle based on preliminary analysis of adverse events (AEs) and pharmacokinetics (PK).

In terms of assessments completed dur the study, cross-sectional imaging was performed every two cycles during therapy. For patients off study therapy, but not starting new therapy, disease assessment was performed according to standard care practices.

Treatment discontinuation criteria included any of the following:
1) Progressive disease by RECIST version 1.1 criteria.
2) Unacceptable toxicity
3) Patient elects to discontinue.
4) Treating physician recommends discontinuation.

### Study Population

Eligible patients (≥18y) had: advanced, unresectable, non-adipocytic STS progressing after 1-5 prior therapies; adequate end-organ function; ECOG performance status 0-1; measurable target lesions (RECIST v1.1); and no prior mTOR or angiogenesis inhibitor treatment. 19 patients were treated. Patients had a median age of 59 years, were predominantly white, and were diagnosed with a soft-tissue sarcoma selected from the group consisting of spindle cell sarcoma, solitary fibrous tumor, leiomyosarcoma, myxofibrosarcoma, undifferentiated pleomorphic sarcoma, melanocytic schwannoma, and pleomorphic rhabdomyosarcoma (Table 1).

TABLE 1
Demographic and Pathologic Baseline.

| Variable | Value |
|---|---|
| Age at first Treatment, years | |
| Median | 59.0 |
| Range | 46.7-72.8 |
| Sex n (%) | |
| Male | 8 (42) |
| Female | 11 (58) |
| Race n (%) | |
| White | 14 (74) |
| Black/African-American | 1 (5) |
| Native Hawaiian/Pacific Islander | 1 (5) |
| Asian | 1 (5) |
| Unknown/Not Reported | 2 (11) |
| Ethnicity n (%) | |
| Hispanic/Latino | 2 (11) |
| No Hispanic/Latino | 16 (84) |
| Unknown/Not Reported | 1 (5) |
| Diagnosis n (%) | |
| Myxofibrosarcoma | 2 (11) |
| Spindle cell sarcoma | 1 (5) |
| Solitary fibrous tumor | 1 (5) |
| Undifferentiated pleomorphic sarcoma | 2 (11) |
| Leiomyosarcoma | 11 (58) |
| Uterine | 3 |
| Non-Uterine | 8 |
| Melanocytic schwannoma | 1 (5) |
| Pleomorphic Rhabdomyosarcoma | 1 (5) |

### Key Inclusion/Exclusion Criteria

Key inclusion criteria include but are not limited to:
>18 years old
Non-adipocytic soft tissue sarcoma
Metastatic or locally advanced without curative therapy, surgery not recommended, and pazopanib indicated.
>=1 measurable target lesions by Response Evaluation Criteria in Solid Tumors (RECIST) version 1.1
Clinical/radiological progression or failure due to toxicity on at least 1 systemic regimen for advanced disease.
No more than 4 prior lines of systemic therapy (no more than 2 prior combination cytotoxic therapies). Neo-adjuvant/adjuvant/maintenance not included.
Adequate end-organ function, including normal left ventricular ejection fraction and QTc<480 msec
Performance status 0/1.

Key exclusion criteria include but are not limited to:
Histology for which pazopanib is not indicated (e.g. adipocytic STS, gastrointestinal stromal tumors, Kaposi's sarcoma).
Prior mTOR or angiogenesis inhibitor.
Uncontrolled/symptomatic central nervous system metastases.
Clinically significant hemorrhage within 6 months.
Active second malignancy, with exceptions.
Uncontrolled diabetes mellitus.
Unstable coronary artery disease, myocardial infarction, or arterial thromboembolic event within 6 months.
Interstitial lung disease, pneumonitis, or pulmonary hypertension.
Uncontrolled hypertension.

### Results

19 patients were treated. Initially, 13 patients received nab-sirolimus on days 1 and 8. Table 2 shows treatment-related adverse events of any grade experienced by >10% of the study population and all grade 3-4 adverse events. DLT included thrombocytopenia (TCP, n=7), decreased WBC/neutrophils (n=2), increased lipase (n=1), and proteinuria (n=1). Due to overlapping AEs or possible nab-sirolimus/pazopanib interaction, further cohorts received 400 mg pazopanib daily and either 30 or 45 mg/m² nab-sirolimus (n=6). There were no DLTs in the 30 mg/m² cohort (n=3). In the 45 mg/m² cohort, 2 out of 3 patients experienced TCP meeting DLT definition.

Grade 3-4 AEs occurring in more than 10% of patients included: TCP (58%), neutropenia (11%), leukopenia (11%), lymphopenia (11%), and diarrhea (11%). Any grade AEs occurring in >50% of patients included: TCP (74%), mucositis (63%), fatigue (58%), and acneiform rash (53%). There was no grade 5 AEs.

TABLE 2
Treatment-related adverse events of any grade experienced
by >10% of the study population and all grade 3-4 adverse events.
Any Grade Adverse Events Experienced
by >10% and All Grade 3-4 Adverse Events.

| | CTCAE | | | | | |
|---|---|---|---|---|---|---|
| | version | | | | | |
| | 5.0 | | | | Total | |
| | Grade | | | | Grade | Grade |
| | (#) | | Total | All | 3-5 | 3-5 |
| Adverse Event Term | 3 | 4 | (n = 19) | % | (n = 19) | % |
| Platelet count decreased | 8 | 3 | 14 | 74% | 11 | 58% |
| Oral mucositis | 1 | | 12 | 63% | 1 | 5% |
| Fatigue | 1 | | 11 | 58% | 1 | 5% |
| Acneiform rash | | | 10 | 53% | 0 | 0% |
| White blood cell decreased | 2 | | 9 | 47% | 2 | 11% |
| Diarrhea | 2 | | 8 | 42% | 2 | 11% |
| Anemia | 1 | | 8 | 42% | 1 | 5% |
| Neutrophil count decreased | 3 | | 7 | 37% | 3 | 16% |
| Dysgeusia | | | 7 | 37% | 0 | 0% |
| Lymphocyte count | 1 | 1 | 6 | 32% | 2 | 11% |
| decreased | | | | | | |
| Nausea | | | 6 | 32% | 0 | 0% |
| Anorexia | | | 6 | 32% | 0 | 0% |
| Epistaxis | | | 6 | 32% | 0 | 0% |
| Hypertension | 1 | | 5 | 26% | 1 | 5% |
| Hypokalemia | | | 5 | 26% | 0 | 0% |
| Ejection Fraction | 1 | | 4 | 21% | 1 | 5% |
| Decreased | | | | | | |
| Alanine aminotransferase | 1 | | 4 | 21% | 1 | 5% |
| increased | | | | | | |
| Aspartate | | | 4 | 21% | 0 | 0% |
| Aminotransferase increased | | | | | | |
| Hypophosphatemia | 1 | | 4 | 21% | 1 | 5% |
| Hypomagnesemia | | | 4 | 21% | 0 | 0% |
| Pruritus | | | 4 | 21% | 0 | 0% |
| Palmar-plantar | | | 4 | 21% | 0 | 0% |
| erythrodysesthesia | | | | | | |
| syndrome | | | | | | |
| Weight loss | | | 3 | 16% | 0 | 0% |
| Headache | | | 3 | 16% | 0 | 0% |
| Maculo-papular rash | | | 3 | 16% | 0 | 0% |
| Alopecia | | | 3 | 16% | 0 | 0% |
| Dyspnea | | | 3 | 16% | 0 | 0% |
| Vomiting | | | 2 | 11% | 0 | 0% |
| Dry mouth | | | 2 | 11% | 0 | 0% |
| Abdominal pain | | | 2 | 11% | 0 | 0% |
| Dizziness | | | 2 | 11% | 0 | 0% |
| Muscle cramp | | | 2 | 11% | 0 | 0% |
| White blood cell decreased | | | 2 | 11% | 0 | 0% |
| Hypocalcemia | | | 2 | 11% | 0 | 0% |
| Hypertriglyceridemia | | | 2 | 11% | 0 | 0% |
| Hyperglycemia | | | 2 | 11% | 0 | 0% |
| Proteinuria | 1 | | 2 | 11% | 1 | 5% |
| Nail Changes | | | 2 | 11% | 0 | 0% |
| Lipase increased | 1 | | 1 | 5% | 1 | 5% |
| Electrocardiogram QT | 1 | | 1 | 5% | 1 | 5% |
| corrected interval | | | | | | |
| prolonged | | | | | | |
| Platelet Count Increased | 1 | | 1 | 5% | 1 | 5% |
| Urinary tract infection | 1 | | 1 | 5% | 1 | 5% |
| Pneumothorax | 1 | | 1 | 5% | 1 | 5% |
| Skin ulceration | 1 | | 1 | 5% | 1 | 5% |

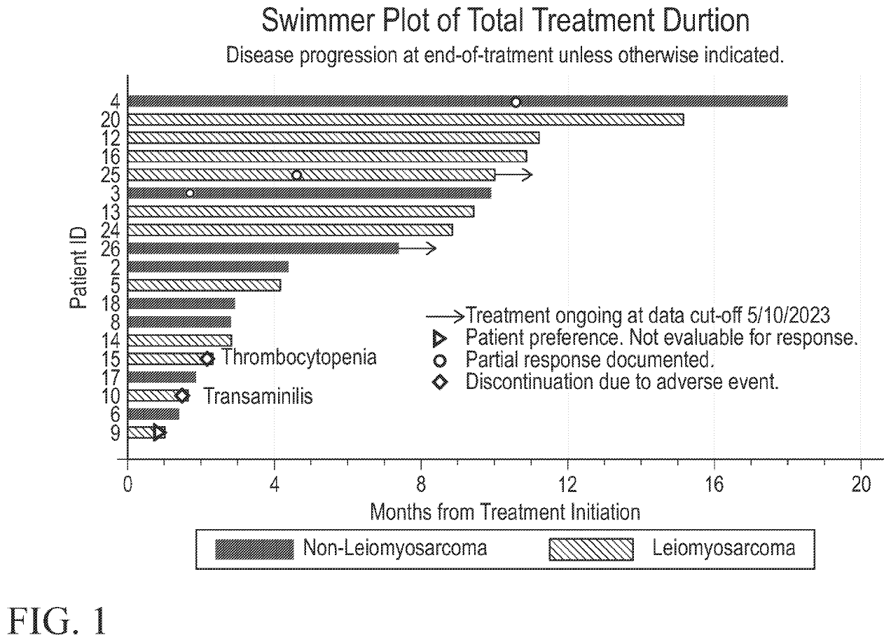

FIG. 1 shows a swimmer plot of total treatment duration for all patients on the study. Disease progression was observed at end-of-treatment unless otherwise indicated, for example a circle marking the time of disease progression. Of 19 treated, 14 (74%) discontinued due to disease progression, 2 (11%) due to AE (TCP-2, transaminitis-1), and 1 (5%) due to death from disease. Two (11%) remain on study as of May 10, 2023.

Table 3 shows the outcomes of treatment by dosing cohort, including the best objective response by RECIST and progression-free survival. Although assessment of activity was not a primary objective of this phase I study, 3- and 6-month progression-free survival were favorable among the 18 evaluable patients, with 13 and 11 patients without progression at these two respective time points. For the leiomyosarcoma subset, 9 and 8 evaluable patients were progression-free at these respective time points.

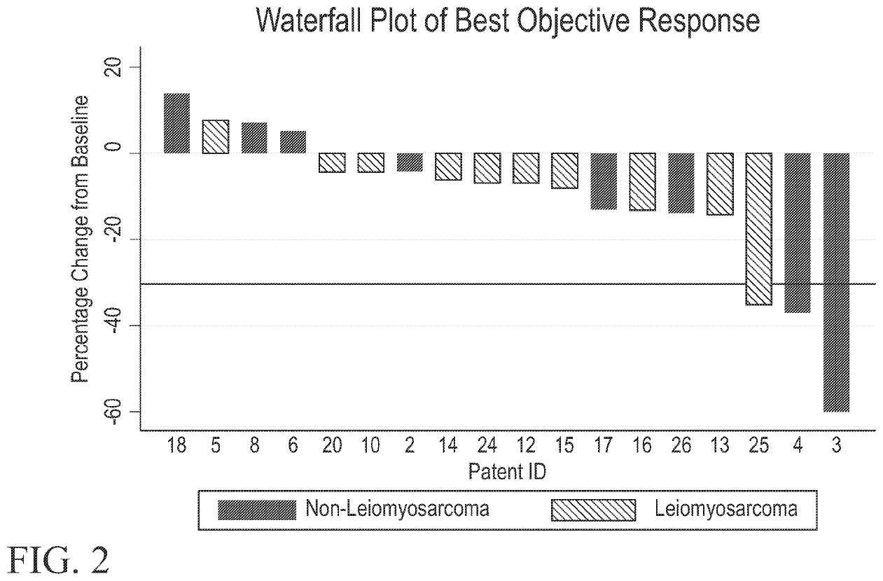

As shown in the waterfall plot in FIG. 2, of the 18 patients evaluable for best response, there were 3 partial responses (leiomyosarcoma, solitary fibrous tumor, spindle cell sarcoma), 13 stable disease, and 2 progressive disease. These response rates represent a significant improvement over response rates of pazopanib treatment alone. Remarkably, among these 18 patients, 2 (11%) maintained stable disease for more than a year.

Clinical Benefit Rates (CBR) at 3 and 6 months were 72% (13/18) and 59% (10/17). Among the leiomyosarcoma subset, CBR at 3 and 6 months were 90% (9/10) and 80% (8/10).

TABLE 3
Outcomes by Dosing Cohort.

| | NAB- | NAB- | | | | | | Best | Progression- |
|---|---|---|---|---|---|---|---|---|---|
| | sirolimus | sirolimus | Pazopanib | | | | Duration | Objective | Free |
| | dose | Frequency | Dose | Patient | | Dose-Limiting | of Therapy | Response | Survival |
| Cohort | (mg/m²) | (Days) | (mg daily) | ID | Histology | Toxicity (Y/N) | (months) | (RECIST) | (months) |
| 0 | 60 | 1.8 | 800 | 2 | MFS | Thrombocytopenia | 4.4 | SD | 4.2 |
| 0 | 60 | 1.8 | 800 | 3 | Spindle cell | WBC decreased, | 9.9 | PR | 9.8 |
| | | | | | sarcoma | Neutropenia | | | |
| −1 | 45 | 1.8 | 800 | 4 | SFT | Neutropenia | 18.0 | PR | 17.7 |
| −1 | 45 | 1.8 | 800 | 5 | uLMS | Lipase elevated | 4.2 | SD | 4.1 |
| −2 | 30 | 1.8 | 800 | 6 | UPS | Thrombocytopenia | 1.4 | PD | 1.2 |
| −2 | 30 | 1.8 | 800 | 8 | UPS | Proteinuria | 2.8 | SD | 2.8 |
| 0B | 60 | 1.8 | 400 | 9 | LMS | Thrombocytopenia | 1.0 | NE | NE |
| 0B | 60 | 1.8 | 400 | 10 | LMS | Thrombocytopenia | 1.6 | SD | 7.4 |
| −1B | 45 | 1.8 | 400 | 12 | LMS | Thrombocytopenia, | 11.2 | SD | 11.1 |
| | | | | | | oral mucositis | | | |
| −1B | 45 | 1.8 | 400 | 13 | LMS | Thrombocytopenia | 9.4 | SD | 9.2 |
| 2B | 30 | 1.8 | 400 | 14 | uLMS | No | 2.8 | SD | 2.7 |
| 2B | 30 | 1.8 | 400 | 15 | LMS | Thrombocytopenia | 2.3 | SD | 8.1 |
| 2B | 30 | 1.8 | 400 | 16 | LMS | Neutropenia, | 10.9 | SD | 10.9 |
| | | | | | | Thrombocytopenia | | | |
| 0C | 30 | 1 | 400 | 17 | Melanocytic | No | 1.8 | PD | 1.2 |
| | | | | | Schwannoma | | | | |
| 0C | 30 | 1 | 400 | 18 | Pleomorphic | No | 2.9 | SD | 0.8 |
| | | | | | RMS | | | | |
| 0C | 30 | 1 | 400 | 20 | LMS | No | 15.2 | SD | 15.2 |
| 1C | 45 | 1 | 400 | 24 | LMS | Thrombocytopenia | 8.9 | SD | 8.9 |
| 1C | 45 | 1 | 400 | 25 | uLMS | No | 10.0 | PR | 10.0 |
| 1C | 45 | 1 | 400 | 26 | MFS | Thrombocytopenia | 7.4 | SD | 7.4 |

LMS: Leimyosarcoma; uLMS: Uterine Leiomyosarcoma; MFS: Myxofibrosarcoma; RMS: Rhabdomyosarcoma; SFT” Solitary Fibrous Tumor; UPS: Undifferentiated Pleomorphic Sarcoma.
RECIST: Response Evaluation Criteria in Solid Tumors version 1.1;
NAB-sirolimus: Nanoparticle Albumin-Bound sirolimus;
NE: Not Evaluable;
PD: Progressive Disease;
PR: Partial Response;
SD: Stable Disease

### CONCLUSIONS

Concurrent administration of pazopanib and nab-sirolimus is feasible and tolerable. Preliminary evidence of activity of the combination was observed. Of the 18 patients evaluable for best response, there were 3 exhibiting partial responses (leiomyosarcoma, solitary fibrous tumor, spindle cell sarcoma), 13 exhibiting stable disease, and 2 exhibiting progressive disease. These results, including those patients having stable disease, are a substantial improvement over existing treatments which exhibited poor response, e.g., about a 6% response rate for single agent pazopanib. Moreover, it was noted that patients treated with a combination of nab-sirolimus and pazopanib exhibited stable disease or partial response for extended periods of time (Table 3).

With dose adjustments, only two patients discontinued therapy due to adverse events. Disease progression was the dominant reason for treatment discontinuation. Recommended Phase 2 doses are pazopanib 400 mg by mouth daily and nab-sirolimus 30 mg/m² intravenously every 21 days. These doses are lower than 800 mg daily and 100 mg/m² on days 1 and 8 every 21 days, respectively, for which these drugs are approved for treatment of soft tissue sarcomas (pazopanib) and PEComas (nab-sirolimus). Dose-limiting toxicities included thrombocytopenia, neutropenia, elevated lipase, and proteinuria. Thrombocytopenia was the most common DLT.

1. A method of treating a soft-tissue sarcoma in an individual in need thereof, the method comprising administering to the individual:
(a) a composition comprising nanoparticles comprising an mTOR inhibitor and an albumin; and
(b) a multi-tyrosine kinase inhibitor,
wherein the multi-tyrosine kinase inhibitor is administered in an amount of about 1 mg to about 2500 mg.

2. (canceled)

3. A method of treating a soft-tissue sarcoma in an individual in need thereof,
wherein the soft-tissue sarcoma is selected from the group consisting of spindle cell sarcoma, solitary fibrous tumor, leiomyosarcoma, myxofibrosarcoma, undifferentiated pleomorphic sarcoma, melanocytic schwannoma, and pleomorphic rhabdomyosarcoma, the method comprising administering to the individual:
(a) a composition comprising nanoparticles comprising an mTOR inhibitor and an albumin; and
(b) a multi-tyrosine kinase inhibitor.

4. The method of claim 3, wherein the soft-tissue sarcoma is uterine leiomyosarcoma.

5. The method of claim 3, wherein the soft-tissue sarcoma is non-uterine leiomyosarcoma.

6. The method of claim 1, wherein the soft-tissue sarcoma is locally advanced, advanced, malignant, advanced malignant, metastatic relapsed, refractory, or resistant to a prior treatment.

7. (canceled)

8. The method of claim 6, wherein the prior treatment comprises a composition comprising nanoparticles comprising an mTOR inhibitor and an albumin.

9. The method of claim 1, wherein the mTOR inhibitor in the mTOR inhibitor nanoparticle composition is administered in an amount of about 10 mg/m² to about 150 mg/m².

10-12. (canceled)

13. The method of claim 1, wherein the mTOR inhibitor nanoparticle composition is administered once or twice out of every 3 weeks.

14-15. (canceled)

16. The method of claim 1, wherein the mTOR inhibitor is sirolimus.

17. The method of claim 1, wherein the average diameter of the nanoparticles in the composition is no greater than about 150 nm.

18. (canceled)

19. The method of claim 1, wherein the weight ratio of the albumin to the mTOR inhibitor in the nanoparticle composition is no greater than about 9:1.

20. The method of claim 1, wherein the nanoparticles comprise the mTOR inhibitor associated with the albumin.

21. (canceled)

22. The method of claim 1, wherein the mTOR inhibitor nanoparticle composition is administered intravenously.

23. The method of claim 1, wherein the multi-tyrosine kinase inhibitor inhibits two or more of the following: vascular endothelial growth factor receptor (VEGFR)-1, -2, or -3, platelet endothelial growth factor receptor (PDGFR)-α or -β, interleukin-2 receptor-inducible T-cell kinase (ITK), leukocyte-specific protein tyrosine kinase (LCK), colony-stimulating factor-1 receptor (c-fms), fibroblast growth factor receptors (FGFR)-1, -3, or -4, or stem cell factor receptor c-Kit.

24. The method of claim 1, wherein the multi-tyrosine kinase inhibitor is pazopanib or a pharmaceutically acceptable salt thereof.

25. (canceled)

26. The method of claim 1, wherein the amount of the multi-tyrosine kinase inhibitor administered to the individual is about 200 mg to about 600 mg.

27. The method of claim 1, wherein the multi-tyrosine kinase inhibitor is administered daily or every other day.

28. The method of claim 1, wherein the multi-tyrosine kinase inhibitor is administered orally.

29. (canceled)

30. The method of claim 1, wherein, when the mTOR inhibitor nanoparticle composition and the multi-tyrosine kinase inhibitor are administered concurrently, sequentially, or simultaneously.

31-32. (canceled)

33. The method of claim 1, wherein the mTOR inhibitor in the mTOR inhibitor nanoparticle composition is administered intravenously in an amount of about 30 mg/m² on day 1 of a 21-day cycle, and wherein the multi-tyrosine kinase inhibitor is administered orally in an amount of about 400 mg daily.

34. (canceled)
